(19) United States
(12) Patent Application Publication
(10) Pub. No.: US 2017/0172909 A1
(43) Pub. Date: Jun. 22, 2017

(54) Method Of Counteracting The Impact Of Chronic Stress On Skin

(71) Applicant: ELC MANAGEMENT LLC, Melville, NY (US)

(72) Inventors: Anke VAN SUMMEREN, Lommel (BE); Caroline Françoise POLLEFLIET, Borgerhout (BE); Hugo A.L. CORSTJENS, Maaseik (BE); Lieve DECLERCQ, Ekeren (BE); Mei YU, Pudong District (CN); Ilse SENTE, Zonhoven (BE)

(21) Appl. No.: 14/977,985

(22) Filed: Dec. 22, 2015

(51) Int. Cl. A61K 8/97; G01N 33/50; A61Q 19/08; A61Q 19/00; A61K 8/63; A61K 8/34

(52) U.S. Cl. CPC A61K 8/97; A61K 8/63; A61K 8/342; A61Q 19/08; A61Q 19/00; G01N 33/5044; G01N 33/5023; A61K 2800/10

(57) ABSTRACT

The present invention is directed to a method for evaluating cosmetic materials for their efficacy in counteracting the effects of chronic stress on skin using a stress-induced premature senescence phenotype skin model. The present invention is also concerned with compositions containing a combination of actives for blocking or reversing the biological impact of chronic stress on the skin together with actives for rebuilding epidermis so as to restore elasticity.

### FIELD OF THE INVENTION

The present invention is related to anti-aging skin care. More particularly, the present invention is directed to methods of identifying cosmetic ingredients demonstrating an efficacy for preventing or counteracting the impact of stress-induced visible signs of fatigued skin. The invention also concerns cosmetic ingredients which can be formulated into skincare products to address the visible signs of chronically stressed or fatigued skin.

### BACKGROUND OF THE INVENTION

With today's busy, modern lifestyle, it is difficult to find the right balance between work and life. This lack of balance often causes stress. It is well-recognized that that chronic stress is associated with prolonged increased levels of cortisol in the blood. It is also commonly accepted that psychological stress is linked to premature aging of the skin. Consumers often identify tired-looking skin or skin fatigue with prematurely aged skin. Self-perceived fatigued skin is characterized by a measurable lack of radiance, visible hyperpigmentation, lines and wrinkles, and skin laxity.

Consumers desire anti-aging treatments which counteract the visible signs of tired skin to revive a more youthful looking, more radiant, even toned, smoother, firmer, and more elastic skin. There is therefore a need for identifying novel ingredients for formulation into cosmetic treatment products for this purpose.

### BRIEF DESCRIPTION OF THE DRAWING FIGURES

FIG. 1a represents a digitized image of untreated explant of stratum corneum on day 0.

FIG. 1b represents a digitized image of untreated explant of stratum corneum on day 9.

FIG. 1c represents a digitized image of cortisol-treated explant of stratum corneum on day 9.

FIG. 2a represents a digitized image of Biwa leaf-treated explant of stratum corneum on day 9.

FIG. 2b represents a digitized image of Biwa leaf- and cortisol-treated explant of stratum corneum on day 9.

FIG. 2c represents a digitized image of Biobenefity-treated explant of stratum corneum on day 9.

FIG. 2d represents a digitized image of Biobenefity- and cortisol-treated explant of stratum corneum on day 9.

FIG. 2e represents a digitized image of IBR Dormin-treated explant of stratum corneum on day 9.

FIG. 2f represents a digitized image of IBR Dormin- and cortisol-treated explant of stratum corneum on day 9.

FIG. 2g represents a digitized image of Juvinity-treated explant of stratum corneum on day 9.

FIG. 2h represents a digitized image of Juvinity- and cortisol-treated explant of stratum corneum on day 9.

FIG. 3 is a graph depicting the staining intensity for p21 in the untreated explants of stratum corneum on day 0 and 9 and the treated explants of stratum corneum on day 9.

FIG. 4 is a graph depicting the staining intensity for progerin in the untreated explants of stratum corneum on day 0 and 9 and the treated explants of stratum corneum on day 9.

FIG. 5a is a graph depicts a comparison of the contractile forces of fibroblasts derived from eyelid and abdominal skins on collagen lattices after 6 hours.

FIG. 5b is a graph depicts a comparison of the contractile forces of fibroblasts derived from eyelid and abdominal skins on collagen lattices after 24 hours.

FIG. 5c represents the AUC contractile forces of fibroblasts derived from eyelid and abdominal skins on collagen lattices.

FIG. 5d represents the maximum contractile forces of fibroblasts derived from eyelid and abdominal skins on collagen lattices.

FIG. 6a is a graph depicting contractile forces of fibroblasts derived from eyelid and abdominal skins on collagen lattices over 6 hours in the presence or absence of cortisol.

FIG. 6b is a graph depicting contractile forces of fibroblasts derived from eyelid and abdominal skins on collagen lattices over 25 hours in the presence or absence of cortisol.

FIG. 6c represents the AUC contractile forces of fibroblasts derived from eyelid and abdominal skins on collagen lattices in the presence or absence of cortisol.

FIG. 6d represents the maximum contractile forces of fibroblasts derived from eyelid and abdominal skins on collagen lattices in the presence or absence of cortisol.

FIG. 7a is a graph depicting contractile forces of Biobenefity-treated and Albizia julibrissin-treated-fibroblasts derived from eyelid and abdominal skins on collagen lattices over 6 hours in the presence or absence of cortisol.

FIG. 7b is a graph depicting contractile forces of Biobenefity-treated and Albizia julibrissin-treated-fibroblasts derived from eyelid and abdominal skins on collagen lattices over 25 hours in the presence or absence of cortisol.

FIG. 7c represents the AUC contractile forces on collagen lattices of fibroblasts derived from eyelid and abdominal skins, untreated, cortisol-treated, or treated with a combination of cortisol with Biobenefity or Albizia julibrisson.

FIG. 7d represents the maximum contractile forces on collagen lattices of fibroblasts derived from eyelid and abdominal skins, untreated, cortisol-treated, or treated with a combination of cortisol with Biobenefity or Albizia julibrisson.

FIG. 8a is a graph depicting contractile forces of Transforming Growth Factor β (TGFβ)-treated and untreated fibroblasts on collagen lattices over 6 hours.

FIG. 8b is a graph depicting contractile forces of TGFβ-treated and untreated fibroblasts on collagen lattices over 24 hours.

FIG. 8c represents the AUC contractile forces on collagen lattices of fibroblasts derived from eyelid and abdominal skins, untreated or treated with TGFβ.

FIG. 8d represents the maximum contractile forces on collagen lattices of fibroblasts derived from eyelid and abdominal skins, untreated or treated with TGFβ.

FIG. 9a is a graph depicting contractile forces of untreated or Taisoh Liquid B Jujube extract-treated fibroblasts on collagen lattices over 6 hours.

FIG. 9b is a graph depicting contractile forces of untreated or Taisoh Liquid B-Jujube extract treated fibroblasts on collagen lattices over 24 hours.

FIG. 9c represents the AUC contractile forces on collagen lattices of fibroblasts derived from eyelid and abdominal skins, untreated or treated with Taisoh Liquid B Jujube extract.

FIG. 9d represents the maximum contractile forces on collagen lattices of fibroblasts derived from eyelid and abdominal skins, untreated or treated with Taisoh Liquid B Jujube extract.

FIG. 10a is a graph depicting contractile forces of untreated or Uplevity-treated fibroblasts on collagen lattices over 6 hours.

FIG. 10b is a graph depicting contractile forces of untreated or Uplevity-treated fibroblasts on collagen lattices over 24 hours.

FIG. 10c represents the AUC contractile forces on collagen lattices of fibroblasts derived from eyelid and abdominal skins, untreated or treated with Taisoh Liquid B Jujube extract.

FIG. 10d represents the maximum contractile forces on collagen lattices of fibroblasts derived from eyelid and abdominal skins, untreated or treated with Taisoh Liquid B Jujube extract.

FIG. 11 is a graph depicting contractile forces of untreated or Juvefoxo-treated fibroblasts on collagen lattices.

FIG. 12 is a graph depicting contractile forces of untreated or NXP-treated fibroblasts on collagen lattices.

FIG. 13 is a graph depicting contractile forces of untreated or Energen-treated fibroblasts on collagen lattices.

FIG. 14 is a graph depicting contractile forces of untreated or Serilesine-treated fibroblasts on collagen lattices.

FIG. 15 is a graph depicting contractile forces of untreated or Raffermine-treated fibroblasts on collagen lattices.

FIG. 16 is a graph depicting contractile forces of untreated or hydrocortisone-treated fibroblasts on collagen lattices.

FIG. 17 is a graph depicting contractile forces of untreated, hydrocortisone-treated fibroblasts, with or without Juvefoxo on collagen lattices.

FIG. 18 is a graph depicting contractile forces of untreated, hydrocortisone-treated fibroblasts, with or without NXP on collagen lattices.

FIG. 19 is a graph depicting contractile forces of untreated or hydrocortisone-treated fibroblasts, with or without Energen on collagen lattices.

FIG. 20 is a graph depicting the effect Solpeptide on Elastin production by Human Dermal Fibroblasts (HDFs).

FIG. 21 is a graph depicting the effect Mitostime on elastin production by HDFs.

FIG. 22 is a graph depicting the effect Uplevity on elastin production by HDFs.

FIG. 23 is a graph depicting the effect Riboxyl on elastin production by HDFs.

FIG. 24 is a graph depicting the effect NXP75 on elastin production by HDFs.

FIG. 25 is a graph depicting the effect TGFB1 on tropelastin production by HDFs.

FIG. 26 is a graph depicting the effect of Decorinyl on elastin production by HDFs.

FIG. 27 is a graph depicting the effect of Eyeseryl on elastin production by HDFs.

FIG. 28 is a graph depicting the effect of Deglysome LYO on elastin production by HDFs.

FIG. 29 is a graph depicting the effect of Gatuline In-tense on elastin production by HDFs.

FIG. 30 is a graph depicting the effect of TGFβ1 on fibrillin production by HDFs.

FIG. 31 is a graph depicting the effect of Milk Peptide on fibrillin production by HDFs.

FIG. 32 is a graph depicting the effect of Mitostime on fibrillin production by HDFs.

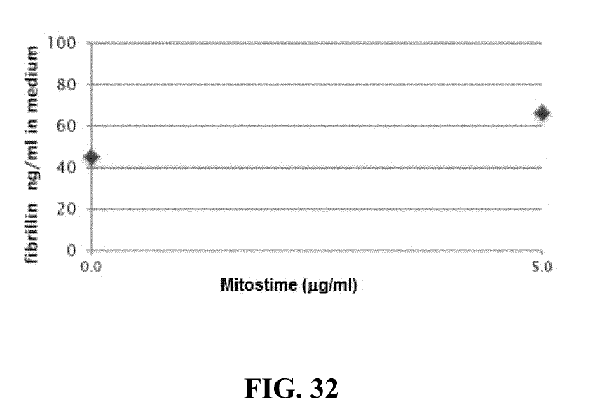

### SUMMARY OF THE INVENTION

The present invention is directed to a method for identifying and evaluating cosmetic materials for their efficacy in counteracting the effects of stress on skin using a stress-induced premature senescence phenotype skin model.

The present invention is also concerned with compositions, regimens and methods for preventing, minimizing, or reversing the biological impact of stress on the skin resulting in prematurely aged skin. The compositions, regimens and methods combine actives which block or reverse the impact of stress on skin with actives which promote the rebuilding of epidermis.

### DETAILED DESCRIPTION OF THE INVENTION

The aging process is accompanied by changes in skin's mechanical properties. These changes, which have been attributed to the altered collagen and elastin organization and density of the skin's extracellular matrix, undesirably affect the skin of the face and the neck which begin to sag due in part to loss of elasticity. Additionally, hyperpigmented spots increase in number and/or become more visible. Fine lines appear and may develop into deeper creases. The glow of youthful, radiant skin fades.

It has been observed that persons who are stressed over prolonged periods of time actually tend to look fatigued. It is commonly believed that psychological stress leads to premature aging, and that such stress advances the onset of aged related oxidative stress and other determinants of cellular senescence. (Epel, E. S. et al. Accelerated telomere shortening in response to life stress. Proc. Natl. Acad. Sci. U.S.A. 101, 17312-17315 (2004)). Such a determinant may be characterized as a biological marker or biomarker. As used herein, a biomarker is a substance, a physiological or morphological characteristic, a gene, or other index, which indicates or which may indicate the presence of a premature senescent stat of aging skin. In vitro senescent cells show a growth arrest with an increased expression of cyclin-kinase inhibitors such as cyclin-kinase inhibitor 1A (p21) which has been used as a marker for senescence (Chen, Q. M. et al. Molecular analysis of H₂O₂-induced senescent-like growth arrest in normal human fibroblasts: p53 and Rb control G1 arrest but not cell replication. Biochem. J. (pt. 1), 43-50 (1998)). Progerin, a truncated form of Lamin A, has been indicated as causing premature aging in Hutchinson-Gilford progeria syndrome and has also been observed to increase in normal cellular ageing. Progerin has been proposed as a further biomarker of ageing (McClintock, D. et al. The mutant form of lamin A that causes Hutchinson-Gilford progeria is a biomarker of cellular aging in human skin. PLoS One. 2007 Dec. 5; 2(12): e1269); Takeuchi, H., et al. Longwave UV light induces the aging-associated progerin. J. Invest. Dermatol. 2013 July:133(7)1857-62.

Aging skin is also associated with a reduction in the level of fibrillins. Fibrillin, encoded by the FBN1 gene, is a glycoprotein that serves two key physiological functions: as a supporting structure that imparts tissue integrity and as a regulator of signaling events that direct cell performance. The structural role of fibrillins is exerted through the temporal and hierarchical assembly of microfibrils and elastic fibers, whereas the instructive role reflects the ability of fibrillins to sequester transforming growth factor β (TGFβ) and bone morphogenetic protein (BMP) complexes in the extracellular matrix (Ramirez, F. et al. Biogenesis and function of fibrillin assemblies. Cell Tissue Res. 2010 January; 339(1): 71-82). The fibrillin rich microfibrillar network of the upper dermis undergoes extensive remodelling resulting in the reduction of fibrillin-1 in photoaged skin. (Watson, R. E. et al., Fibrillin-rich microfibrils are reduced in photoaged skin: Distribution at the dermal-epidermal junction. J. Invest. Dermatol. 1999; 12(5): 782-7; Watson, R. E. et al. A short-term screening protocol using fibrillin-1 as a reporter molecule for photoaging repair agents. J. Invest. Dermatol. 2001; 116(5): 672-8).

Elastin, another protein of the extracellular matrix, is responsible for the skin's elasticity and resilience. Elastin is secreted by fibroblasts as the soluble precursor tropoelastin that is subsequently cross-linked into insoluble elastin. In tissue, elastin is further complexed with microfibrils to form the elastic fibers. These elastin fibers are enriched in the dermis where they impart skin flexibility, extensibility and recoil. However, as skin ages, the elastin becomes disorganized and thus less functional leading to sagging skin. Additionally, with age, there is a general reduction in biosynthetic capacity of fibroblasts and a progressive disappearance of elastic tissue in skin (Jenkins, G. Molecular mechanisms of skin ageing. Mech. Ageing Dev., 123, 801-810 (2002).

A diminished level of contractility of dermal fibroblasts has also been associated with senescence (Knott, A. et al., Decreased fibroblast contractile activity and reduced fibronectin expression are involved in skin photoaging, Journal of Dermatological Science, 58 (2010) 75-77). It has been posited that a decrease in the contractile forces of the fibroblasts from the eyelid might contribute to the development of droopy eyelids. The literature indicates that dermal fibroblasts lose their contractile forces with age due to a decrease in myosin light chain phosphorylation enzymes (Fujimua, T., et al. Loss of contraction force in dermal fibroblasts with aging due to decrease in myosin light chain phosphorylation enzymes. Arch. Pharm. Res. 34, 1015-1022 (2011). These contractile forces are related to the elasticity of the skin. Consequently, a decrease in contractile forces would be expected to lead to less elastic skin or elastic fatigued skin, increased laxity, and eventually the development of wrinkles.

Aging skin is further characterized by a reduction in the number of cellular layers in the skin. This is exemplified by epidermal atrophy, a decreased thickness of the dermis and/or a decrease in the amount of subcutaneous fat, all of which may also result in wrinkles. Recent studies revealed that dermal fibroblasts undergo morphological changes and cell body collapse in both chronically aged and photo-aged skin (C. Schulze, et al., Stiffening of human skin fibroblasts with age, Biophysical Journal, 99 (2010) 2434-2442; A. Knott, et al., Decreased fibroblast contractile activity and reduced fibronectin expression are involved in skin photoaging, Journal of Dermatological Science, 58 (2010) 75-77). While young dermal fibroblasts exhibit a sufficient capacity to adequately maintain the homeostasis of the extra cellular matrix (ECM), (photo)-aged fibroblasts not only display a decrease of their synthetic activity but are also reduced in number (B. A. Gilchrest, Age-associated changes in the skin, Journal of the American Geriatrics Society, 30 (1982) 139-143).

A fibroblast-populated collagen lattice (FPCL), type of in-vitro dermal equivalent model, has been used to investigate the biological mechanisms of mechanical properties in fibroblasts by evaluating the capacity of fibroblasts to contract the collagen gel of the lattice as evidenced by a reduced lattice area. Biological mechanisms investigated include wound contraction, and also the effects of various compounds aimed at stimulating the rate of contraction or reducing the rate of contraction (T. Tateshita, et al., Effects of collagen matrix containing transforming growth factor (TGF)-beta(1) on wound contraction, Journal of Dermatological Science, 27 (2001) 104-113).

It is well-established that as a response to psychological stress, the stress hormone, cortisol, a glucocorticoid steroid hormone produced by the adrenal cortex, is released into the blood as part of the “fight-or-flight” mechanism. This mechanism causes our bodies to become mobilized and ready for action. This kind of stress is defined as “eustress” or good stress. However, if the level of cortisol in the blood does not normalize and return to baseline, there will be a buildup of cortisol which may result in negative effects on the mind and body. This type of stress is defined as “distress” or bad stress. It has been observed that an increase in the cortisol levels in the blood has the potential to either enhance or to undermine psychobiological resilience to oxidative damage, depending on the body's prior exposure to chronic psychological stress (Aschbacher, K. et al. Good stress, bad stress and oxidative stress: insights from anticipatory cortisol reactivity. Psychoneuroendocrinology. 38, 1698-1708 (2013)).

In humans, the amount of cortisol present in the blood undergoes diurnal variation; the level peaks in the early morning, at approximately 8 a.m., and reaches its lowest level between about midnight and 4 a.m., or three to five hours after the onset of sleep. Changed patterns of serum cortisol levels have been observed in connection with abnormal ACTH levels, clinical depression, psychological stress, and physiological stressors such as hypoglycemia, illness, fever, trauma, surgery, fear, pain, physical exertion, or temperature extremes. It has been observed, in both rodents and humans, that the induction of psychological stress is associated with increased endogenous glucocorticoid production; the administration of systemic glucocorticoids adversely affects barrier homeostasis and epidermal cell proliferation in rodents (Denda, M. et al., Stress alters cutaneous permeability barrier homeostasis, Am. J. Physiol. Regul. Integr. Comp. Physiol., 278(2000) R367-R372). Other investigators have also shown that antagonism of glucocorticoid action reverses a psychological stress-induced delay in wound healing in rodents (D. A. Padgett, et al., Restraint stress slows cutaneous wound healing in mice, Brain, Behavior, and Immunity, 12 (1998) 64-73). It also has been observed that the capacity of fibroblasts to contract collagen fibrils in a three-dimensional collagen lattice (FPCL) is inhibited in a dose-dependent fashion by hydrocortisone (Coulomb, B, et al., The contractility of fibroblasts in a collagen lattice is reduced by corticosteroids. J. of Invest. Dermatol., 82 (1984) 341-344).

As chronic exposure to cortisol may accelerate various biological processes leading to prematurely aged skin or fatigued skin, there remains a need for the further exploration of stress-induced changes in mechanical properties of human dermal fibroblasts (HDFs) and means for visibly reversing the development of these stress-induced changes.

The present invention therefore is directed to a model which mimics stress-induced fatigue of the skin. More specifically, the invention concerns a method of using a stress-induced premature senescence phenotype skin model to identify and evaluate novel cosmetic materials for their efficacy in preventing, minimizing or reversing development of the stress-induced premature senescence phenotype, and formulating such novel cosmetic materials identified as demonstrating such efficacy into cosmetic products for rebuilding epidermis and rejuvenating skin.

According to one embodiment of the invention, a method for identifying a cosmetic material having an efficacy for reversing a stress-induced premature senescence phenotype associated with the appearance of fatigued skin comprises:

(a) providing a dermal equivalent skin model;

(b) incubating the dermal equivalent skin model of (a) with a stress-inducing ingredient in an amount and for a time sufficient to induce a premature senescence phenotype in the dermal equivalent skin model;

(c) incubating the dermal equivalent skin model of (b) with a test material; and

(d) ascertaining whether the test material has an efficacy for reversing the premature senescence phenotype in the skin model.

According to another embodiment of the invention, a method for identifying a cosmetic material having an efficacy for preventing or minimizing development of a stress-induced premature sensescence skin type associated with the appearance of fatigued skin comprises:

(a) providing a dermal equivalent skin model;

(b) treating the dermal equivalent skin model of (a) with a test material;

(c) treating the dermal equivalent skin model of (b) with a stress-inducing ingredient in an amount and for a time sufficient to have induced a premature senescence phenotype in a dermal equivalent skin model in the absence of the test material; and

(d) ascertaining whether the test material has an efficacy for preventing or minimizing development of the premature senescence phenotype in the dermal equivalent skin model.

Skin models useful in carrying out the present invention may be selected from, for example, an in vitro model comprising human dermal fibroblasts (HDFs), an ex vivo model comprising HDFs, or a fibroblast populated collagen lattice.

The stress-induced premature senescence phenotype at the cellular level is characterized by the presence of a biomarker which may be selected from an increase in expression of p21 in fibroblasts, an increase in expression of progerin in fibroblasts, a decrease in elastin production in fibroblasts, a decrease in fibrillin production in fibroblasts, a decrease in fibroblast contractility, a decrease in the number of skin layers, as exemplified by epidermal atrophy, decreased thickness of dermis or decreased amount of subcutaneous fat, or a combination of any two or more thereof. While not wishing to be bound by any particular theory, it is believed that the premature senescence phenotype at the cellular level is associated with visual effects on the skin, i.e., signs of fatigued skin, including the appearance of wrinkles in the skin, hyperpigmented skin, loss of subcutaneous fat, skin laxity, and reduced skin radiance.

Stress-inducing ingredients useful in the present invention include any ingredient which is capable of inducing the premature senescence phenotype in a dermal equivalent skin model containing HDFs. A preferred stress-inducing ingredient useful in the present invention is corti sol.

The stress-inducing ingredient, for example, cortisol, is introduced to the dermal equivalent skin model in an amount and for a time effective to induce the premature senescence phenotype in the skin model. For example, the stress-inducing ingredient may be used topically or systemically in the range of from about 0.000001% to about 5%, including all amounts inbetween, such as about 0.1%, by total weight of the composition applied, and for a time in the range of from about 1 hour to about 72 hours.

A test material is introduced to the dermal equivalent skin model in an amount and for a time effective to ascertain whether the test material has an efficacy for preventing, minimizing or reversing development of the stress-induced premature senescence phenotype in the skin model. For example, the test material may be used in the range of from about 0.0001 to about 5%, such as from about 0.001 to about 0.5%, including all amounts inbetween, by total weight of the composition applied systemically, and for a time in the range of from about 1 hour to about 7 days. The invention also concerns compositions which comprise a novel combination of complimentary active ingredients designed to address signs of skin fatigue, including, wrinkles in skin, hyperpigmented skin, skin laxity, reduced presence of subcutaneous fat, and reduced skin radiance, emanating from a multiplicity of biological pathways and/or by a multiplicity of biological mechanisms.

In accordance with the invention, there is provided a composition for preventing, minimizing or reversing a biological impact of stress on skin, the composition comprising

(a) at least one cosmetic material demonstrating a protecting efficacy against development of a premature sensescence phenotype characteristic of fatigued skin; and

(b) at least one cosmetic raw material demonstrating an efficacy for rebuilding epidermis, wherein a combination of (a) and (b) results in a restored elasticity of the skin.

Cosmetic material (a) may be selected from those which protect against stress-induced enhanced expression of p21 or progerin in fibroblasts, decreased fibroblast contractility, decreased elastin production in fibroblasts, decreased fibrillin production in fibroblasts, and a decreased number of skin layers, as exemplified by one or more of epidermal atrophy, decreased thickness of dermis and decreased amount of subcutaneous fat. Cosmetic material (b) may be selected from those which promote the production of fibrillin, elastin or both in HDFs.

The composition preferably comprises a novel combination of complimentary active ingredients which is designed to address skin fatigue emanating from a multiplicity of biological pathways and/or by a multiplicity of biological mechanisms. Such compositions, which may take the form of aqueous-containing solutions, dispersions or emulsions, combine ingredients found to prevent, minimize or reverse a stress-induced senescence phenotype in skin with ingredients which promote the rebuilding of the epidermis, including, but not limited to ingredients which stimulate the production of elastin and/or fibrillin.

The invention further comprises treating skin for improvement by applying to the skin in need thereof the compositions of the invention. In a accordance with the invention, a method for improving the appearance of fatigued skin is provided, the method comprising

(a) applying to skin in need of such improvement at least one cosmetic material demonstrating an efficacy for protecting against or reversing development of a stress-induced premature senescent phenotype associated with appearance of fatigued skin; and

(b) applying to skin in need of such improvement at least one cosmetic material demonstrating an efficacy for rebuilding epidermis, in particular, an efficacy for promoting elastin production, fibrillin production, or both; wherein (a) and (b) may be applied to skin simultaneously or sequentially in any order to restore elasticity to the skin.

More specifically, step (a) comprises applying to the skin a cosmetic material demonstrating an efficacy for one or more of:

(1) preventing or reversing increased expression of p21 or progerin in fibroblasts,

(2) preventing or reversing decreased fibroblast contractility,

(3) preventing decreased elastin production in fibroblasts,

(4) preventing decreased fibrillin production in fibroblasts, and

(5) preventing or reversing a decreased number of skin layers; and step (b) comprises applying to the skin a cosmetic material demonstrating an efficacy for one or more of increasing synthesis of elastin and increasing synthesis of fibillin.

The compositions may be applied in the forms mentioned herein, as part of skin care regimens. For example, a composition according to the invention may contain both the ingredients for protecting against development of the stress-induced senescence phenotype and ingredients for rebuilding epidermis. The composition may be applied to skin daily, such as, morning and evening. Alternatively, a composition containing ingredients for protecting skin against the development of the premature senescence phenotype may be applied to skin separately from a composition containing epidermis rebuilding ingredients as part of a daily regimen, or the compositions may be applied on alternating days. As a further example, the compositions may take the form of a day cream or a night cream. In another example, active ingredients may be delivered in a composition having a texture that provides sensorial cues to enhance the perceived benefits of relieving stress-induced fatigue of the skin. For example, the composition to be applied in the morning may have a refreshing sensation and a frosted appearance. Such compositions may include lifting polymers to enhance the immediate perception of stress relief, including smoothing and tightening the skin's appearance. In the evening, the composition may have a satin-like texture and may be delivered from a heated dispenser to enhance the immediate perception of stress relief by providing a feeling of warmth and comfort. The compositions may be applied after cleansing the skin. The compositions may be applied to the skin under or over skin care products, such as foundations or other color cosmetics or incorporated into such skin care products or into foundations or other color cosmetics.

### Examples

As used herein, percentages are by weight, unless otherwise indicated.

### Example 1—Evaluation of Test Materials for Efficacy in Reversing Cortisol-Induced Premature Senescence in Ex Vivo Skin

### Explants Preparation

Thirty three skin explants from the abdominal tissue of a female Caucasian donor, age 61 years, of an average diameter of 10 mm (±1 mm) were prepared. The explants, divided into 12 batches, as shown in Table 1 below, were treated with the following actives for their efficacy in reversing cortisol-induced premature senescence: Biwa leaf (Eribotraya japonica, containing saponins, ursolic acid, olianolic acid, maslinic acid, cyanophore glycosides, amygdalin, and tannins); Biobenefity (Cynara scolymus or artichoke leaf extract); IBR Dormin (Narcissus bulb extract); and Juvinity (Geranylgeranyl-2-propanol (6, 10, 14, 18-tetramethylnonadeca-5, 9, 13, 17-tetraen-2-ol, a derivative of isoprene, a complex lipid).

TABLE 1

| | No. of | | |
|---|---|---|---|
| Batch | explants | Treatment | Sampling time |
| B0 | 3 | — | day 0 |
| B | 3 | — | day 9 |
| Cortisol | 3 | Formula with 0.1% | day 9 |
| | | cortisol | |
| Biwa leaf | 3 | Biwa leaf at 0.5% | day 9 |
| | | w/v | |
| Biwa leaf + | 3 | Biwa leaf at 0.5% | day 9 |
| Cortisol | | w/v + Formula | |
| | | with 0.1% cortisol | |
| Biobenefity | 3 | Biobenefity at | day 9 |
| | | 0.5% w/v | |
| Biobenefity + | 3 | Biobenefity at | day 9 |
| Cortisol | | 0.5% w/v + | |
| | | Formula with 0.1% | |
| | | cortisol | |
| IBR Dormin | 3 | IBR Dormin at | day 9 |
| | | 0.1% w/v | |
| IBR Dormin + | 3 | IBR Dormin at | day 9 |
| Cortisol | | 0.1% w/v + | |
| | | Formula with 0.1% | |
| | | cortisol | |
| Juvinity | 3 | Juvinity at 0.5% | day 9 |
| | | w/v | |
| Juvinity + Cortisol | 3 | Juvinity at 0.5% | day 9 |
| | | w/v + Formula | |
| | | with 0.1% cortisol | |

### Product Application

The explants were treated with the different active ingredients by refreshing the culture medium, in which the ingredients were dissolved, on days 0, 1, 2, 5, 6, 7 and 8. The formula with 0.1% cortisol was applied topically on days 2, 5, 6 and 7. The control explants BO and B did not receive any treatment.

### Sampling

On day 0, the three explants from the batch BO were collected and cut in two parts. One half was fixed in buffered formalin, and the other half was frozen at −80° C. On day 9, three explants from all other batches were collected and processed in the same way.

### Histological Processing

After fixation for 24 hours in buffered formalin, the samples were dehydrated and impregnated in paraffin using a Leica TP 1010 dehydration automat. The samples were then embedded using a Leica EG 1160 embedding station. 5-μm-thick sections were made using a Leica RM 2125 Minot-type microtome, and the sections were then mounted on Superfrost® histological glass slides.

### Assessment of Anti-Senescence Activity of Four Products on Human Ex Vivo Skin Explants

The frozen samples were cut into 7-μm-thick sections using a Leica CM 3050 cryostat. Sections were then mounted on Superfrost® plus silanized glass slides. The microscopical observations were made using a Leica DMLB or Orthoplan microscope. Pictures were digitized with a numeric DP72 Olympus camera with CellD storing software.

### General Morphology

The observation of the general morphology was realized after staining of paraffinized sections according to Masson's trichrome, Goldner variant.

### Progerin Immunostaining

Progerin immunostaining was realized on paraffinized sections with a mouse anti-progerin, monoclonal antibody, clone 13A4 (Sigma ref SAB4200272), at 1/200, during 1 hour at room temperature with a biotin/streptavidin amplifier system and revealed using the vector VIP peroxidase (HRP) Substrate kit (Vectorlabs). The immunostaining was assessed by microscopical observation.

### p21 Immunostaining

p21 immunostaining was realized on paraffinized sections with a mouse anti-p21, monoclonal antibody, clone F-5 (Santa Cruz ref sc-6246), at 1/50 eme, during 1 night at 4° C. with a biotin/streptavidin amplifier system and revealed in vector VIP peroxidase (HRP) Substrate kit (Vectorlabs). The immunostaining was assessed by microscopical observation.

### Results

### General Morphology

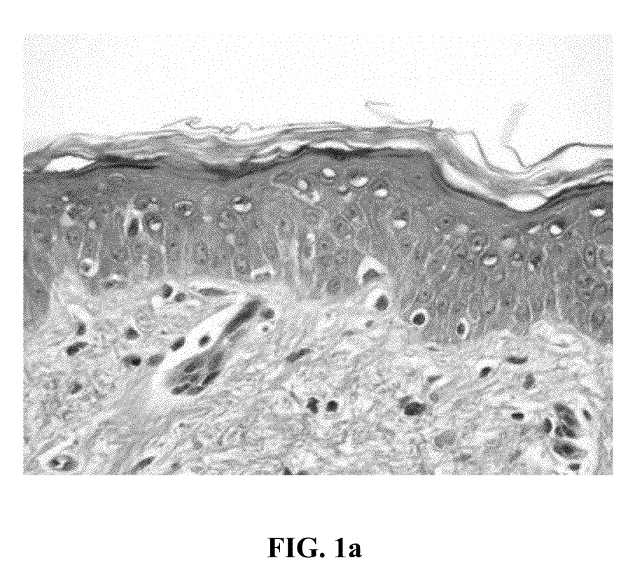

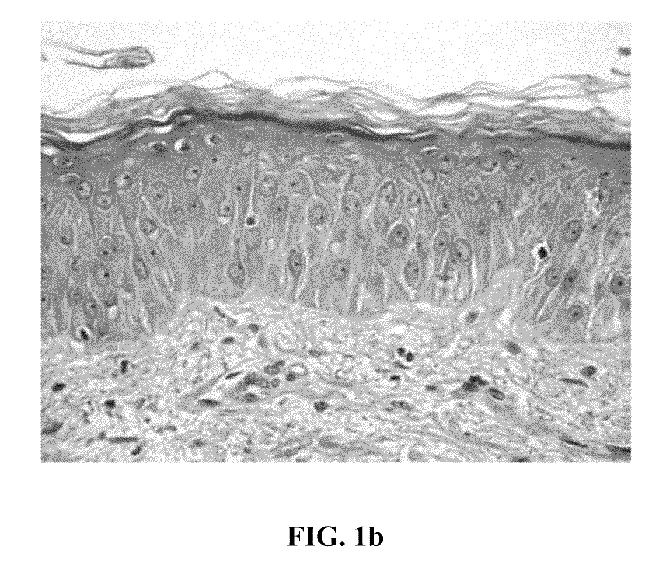

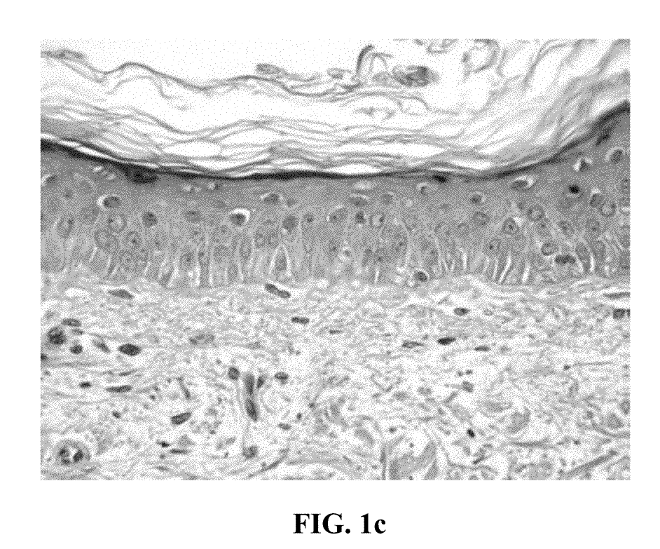

On day 0, the stratum corneum was moderately thick, slightly laminated, moderately keratinized on surface with a slight parakeratosis. The epidermis presented 4 to 5 cellular layers with a normal morphology. The relief of the dermal-epidermal junction was weak. The papillary dermis presented thick collagen bundles forming a relatively dense network which was well-cellularized. On day 9, the general morphology of the untreated explants was very similar to that observed on day 0. Long term treatment with 0.1% cortisol during 7 days (from day 2 to day 9) induced a moderate epidermal atrophy with a decrease in the number of cellular layers (FIG. 1).

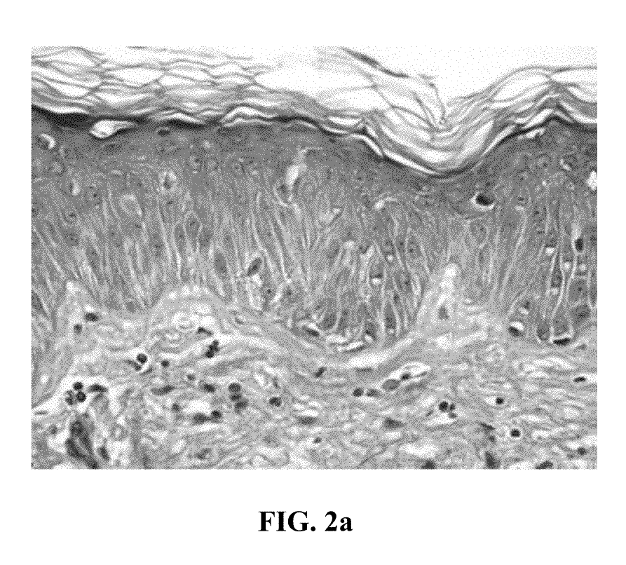

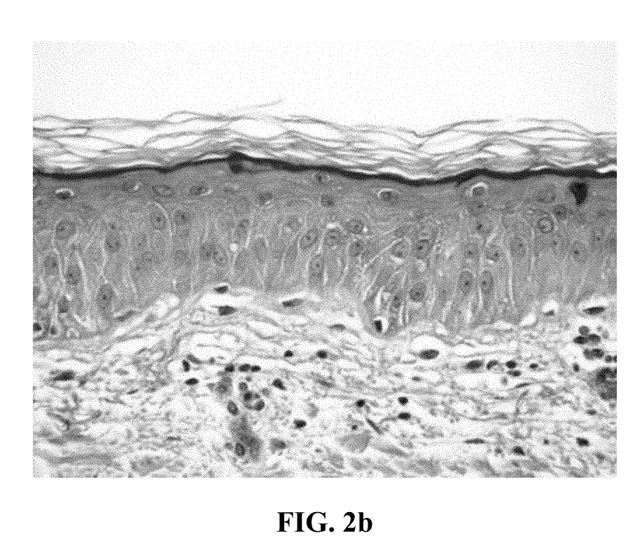

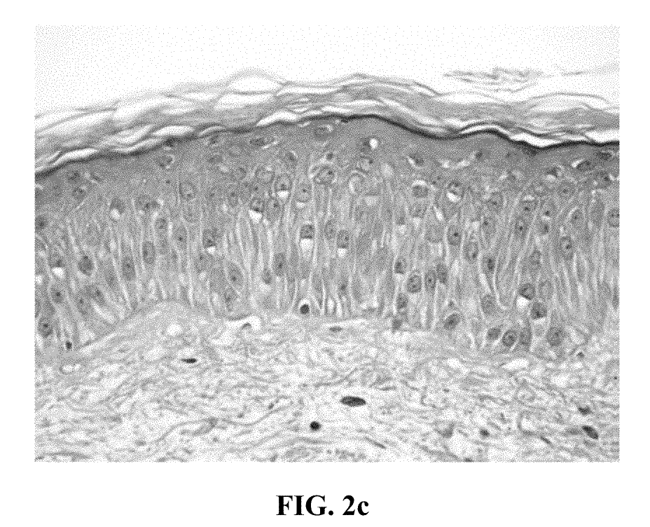

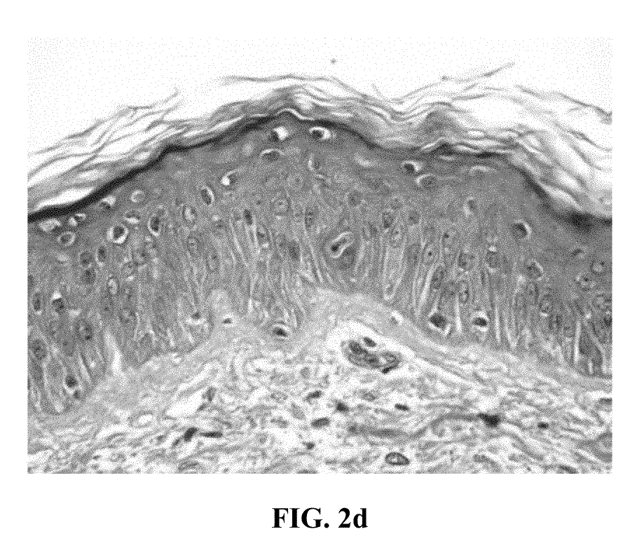

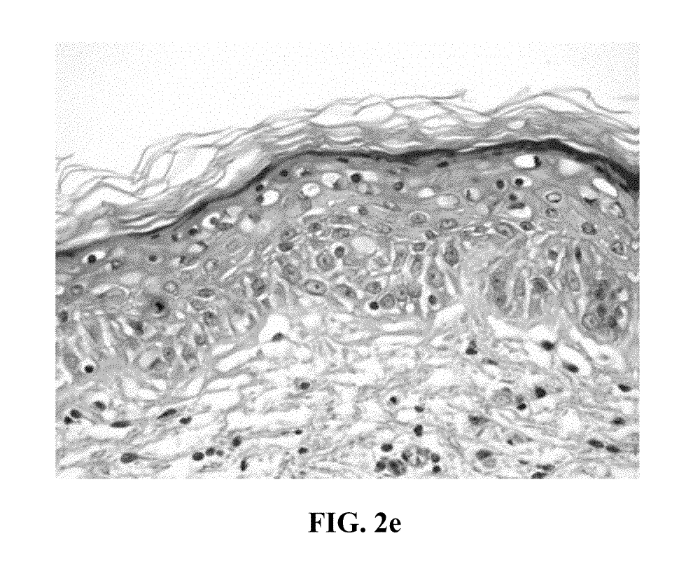

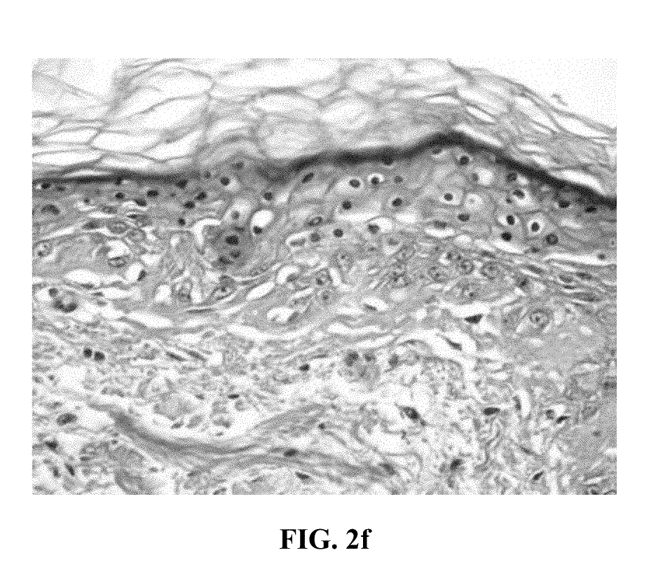

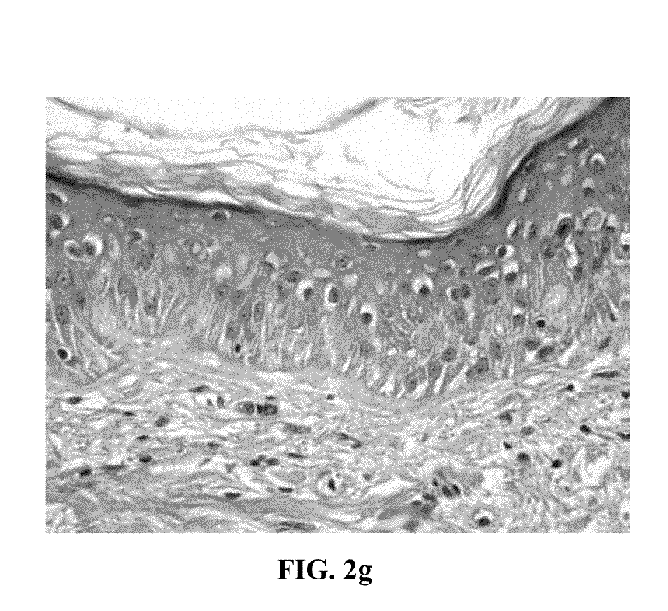

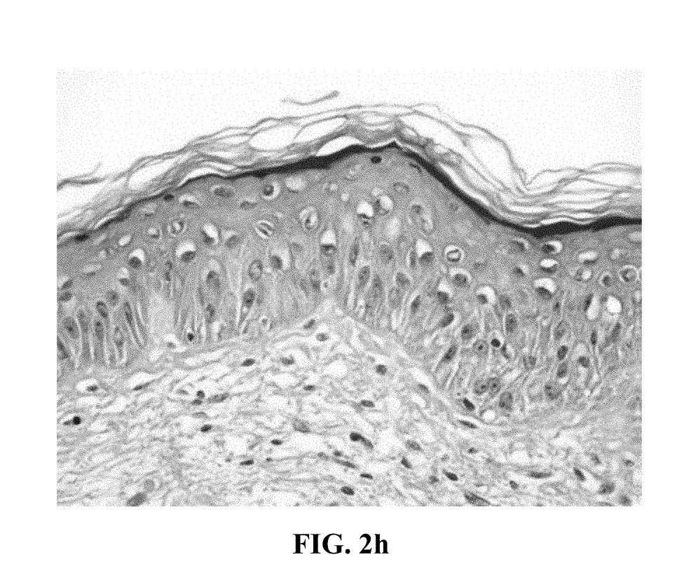

Treatment with the cosmetic raw materials in the absence of the cortisol application, showed no significant difference in morphology for Biwa Leaf and Biobenefity. The explants treated with IBR-Dormin and Juvinity showed an altered morphology, with pycnotic nuclei and cellular spongiosis (FIG. 2). In the presence of cortisol-stress, both Biwa Leaf and Biobenefity caused a slight increase of the epidermal thickness under these conditions, and thus partially reduced the effect of the cortisol treatment. Both IBR-Dormin and Juvinity did not have any beneficial effect on the cortisol-induced morphology under these conditions (FIG. 2).

### P21

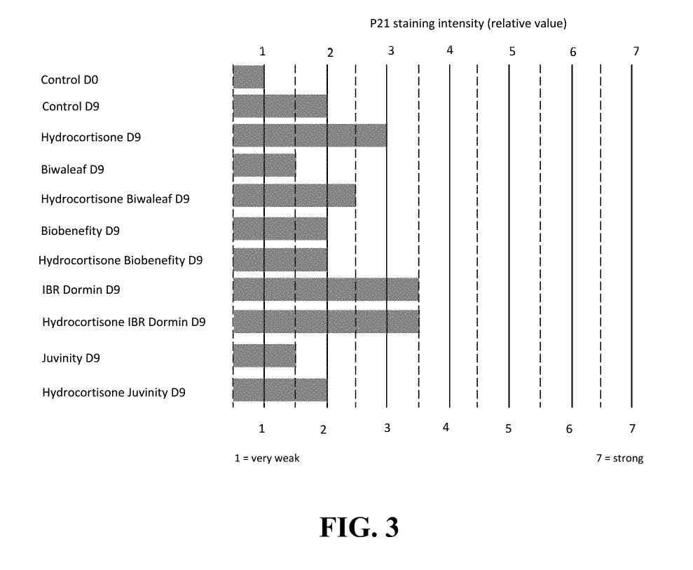

The evaluation of the p21 immunostained pictures was based on both the number of cells that were stained as well as the staining intensity in each cell. This resulted in a semi-quantitative grading. The staining intensity of p21 increased due to the treatment with cortisol from very weak to moderate (FIG. 3). Biobenefity, Juvinity and Biwa Leaf were able to partially reduce this cortisol induced increase in p21 staining intensity. Strongest protective activity was found for Biobenefity, which reduced p21 staining intensity to the level measured in the control without cortisol stress. IBR Dormin showed highest staining intensity, independent of the presence of corti sol.

### Progerin

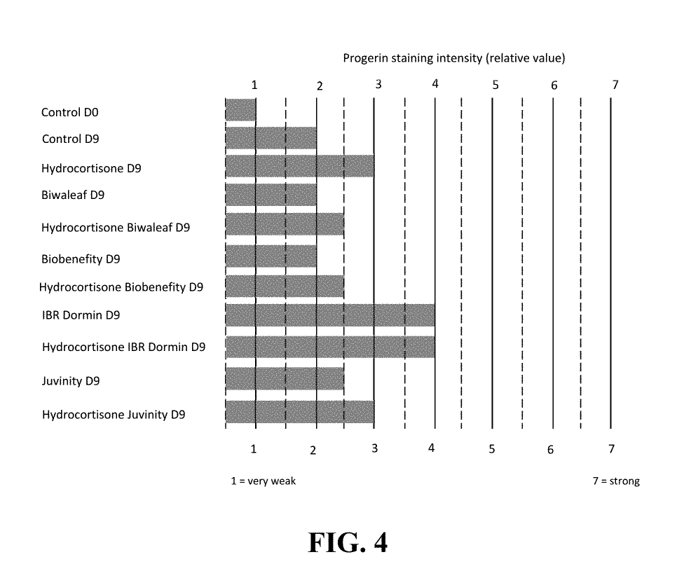

Progerin staining intensity was measured in a similar way as for p21. The staining intensity of progerin increased from weak to moderate due to the treatment with cortisol (FIG. 4). Biwa Leaf and Biobenefity were able to partially reduce this cortisol induced increase in progerin staining intensity. Juvinity and IBR Dormin did not show a beneficial effect on progerin immunostaining intensity under these conditions.

### CONCLUSION

Seven days of treatment of senescent phenotype ex vivo skin with 0.1% cortisol caused morphological changes resulting in increased immunostaining intensity of p21 and progerin. As both Biwa Leaf and Biobenefity partially reduced the cortisol-induced modifications, each could be considered for use as anti-ageing compounds in cosmetic formulations to counteract the impact of psychological stress (i.e., fatigued skin).

### Example 2—Evaluation of Test Materials for Efficacy in Reversing Cortisol-Induced Decrease in Contractile Forces of Fibroblasts Populated on Collagen Lattice

In this study, the GlaSbox® system was used to analyze the effect of cortisol, with and without test materials, on the contractile forces generated by fibroblasts populating collagen lattices. The GlaSbox® device differs from other collagen lattice systems in that it uses a fixed, non-floating collagen lattice where the diameter of the collagen lattice remains constant but electrodes measure the actual contractile forces exerted by the cells on the collagen lattice.

Fibroblasts were obtained from eyelid and abdominal skin of Chinese female donors. The effect of Biobenefity and Albizia julibrissin on the contractile forces of fibroblasts originating from eyelid, with or without exposure to cortisol (250 ng/ml), were evaluated.

Biobenefity, available from Ichimaru Pharcos, is an extract from the leaves of Cynara Scolymus (artichoke). It is said that Biobenefity controls the activity of nuclear factor kappa-light-chain enhancer of activated B cells (NF-κB) and protects the skin from cellular responses to stress such as photoaging. Previously, the inventors observed that Biobenefity induced a trend toward decreasing protein expression of the cyclin-kinase inhibitor p21 in H₂O₂-induced premature senescence in normal human dermal fibroblasts (HDFs) in vitro (data not shown). It also has been observed that H₂O₂-induced premature senescence is accompanied by reduced HDF proliferation. Biobenefity showed a trend toward counteracting this effect as well (data not shown). Biobenefity also was able to prevent the changes in epidermal morphology and increase of p21 and progerin in ex vivo skin repeatedly exposed to cortisol (see Example 1 above).

Albizia julibrissin extract, available from Sederma is said to protect and repair protein structures damaged by glycation, helping to maintain cell viability under conditions of (glycoxidative) stress. Previously, the inventors observed that Albizia julibrissin, at 0.02-0.005% (w/v), decreased protein expression of p21 in H₂O₂-induced premature senescence in normal HDF (data not shown).

### Preparation of Collagen Lattices Under Tension and Measurement of the Isometric Forces

Two fibroblast cell types, one originating from eyelid and the other one from abdominal skin tissue, were purchased from Tebu-Bio (Boechout, Belgium). These cell types were isolated from different donors, each being a 40 year old Chinese woman. The fibroblasts were embedded three-dimensionally in hydrated collagen gel lattices. The gel mixture, composed of 6 volumes of 1.76× (DMEMc, NaHCO3, NaOH, antibiotics), 3 volumes rat tail type I collagen (2 mg/ml), and 1 volume of cellular suspension (8×10⁵ cells/ml), was poured into the rectangular culture plate of the GlaSbox® and polymerized in less than 30 minutes at 37° C. Immediately after lattice formation, actives were added in the cell culture medium. The GlaSbox® was then placed into a humidified incubator at 37° C., and force measurements were initiated, after 30 minutes of stabilization, for 24 hours. The forces are expressed as arbitrary units.

### Calculations and Statistical Analysis

Each Glasbox® curve was fitted with GraphPad Prism® software to determine the area under the curve (AUC) and the maximum of contraction (Max). The area under the curve provides data on the global contraction of fibroblasts during the experiment. Maximum contraction corresponds to the plateau of the fitted curve. Data are expressed as mean±standard deviation. The measurement of contractile forces was analyzed by means of a variance analysis with two factors (group versus control and time). This was followed by a Fisher post-hoc test. A p value less than 0.05 was considered significant.

### Results

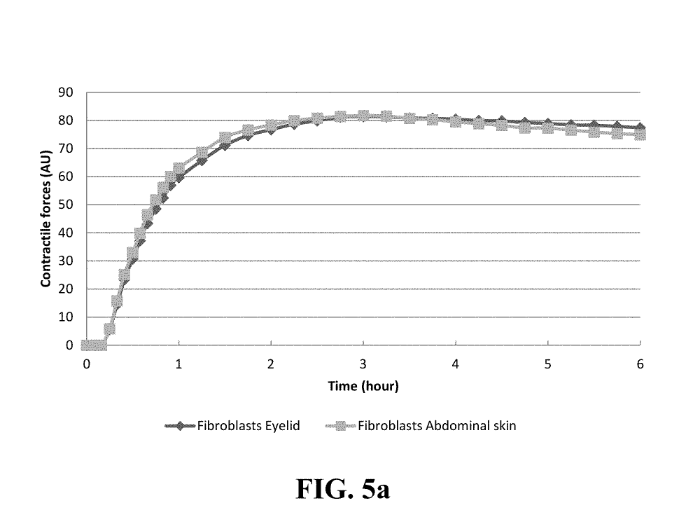

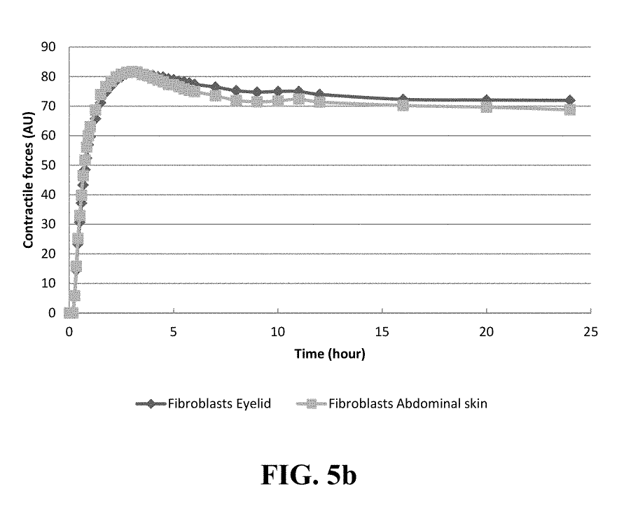

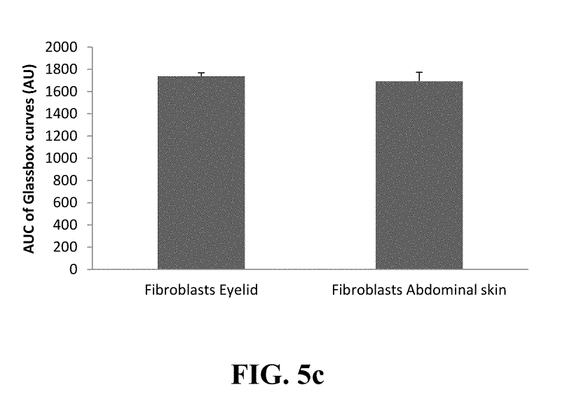

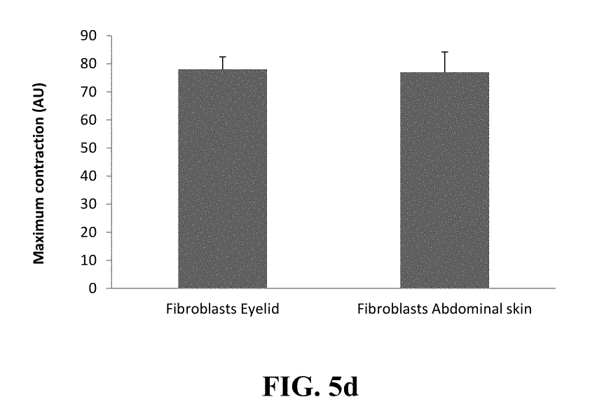

FIGS. 5a, 5b, 5c and 5d depict the contractile forces of fibroblasts exerted on the collagen lattice as a function of time. Initially, there was observed an almost linear increase of the contractile forces. The maximum and/or plateau value is reached at about 3 hours. No significant difference was observed between the contractile forces of fibroblasts from eyelid or abdominal tissue under these experimental conditions.

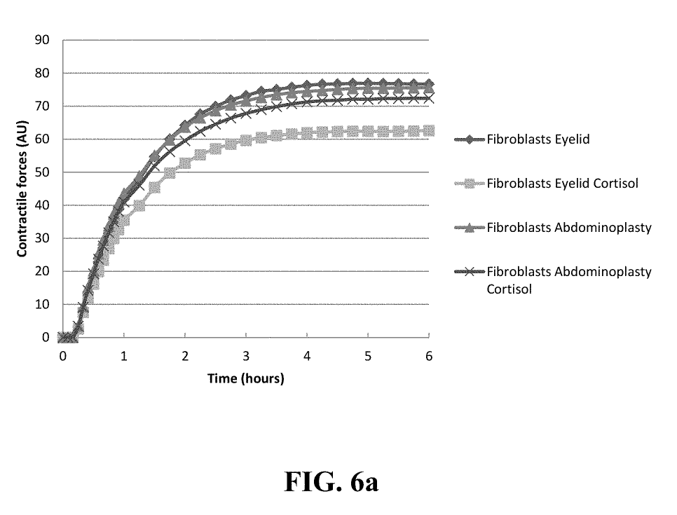

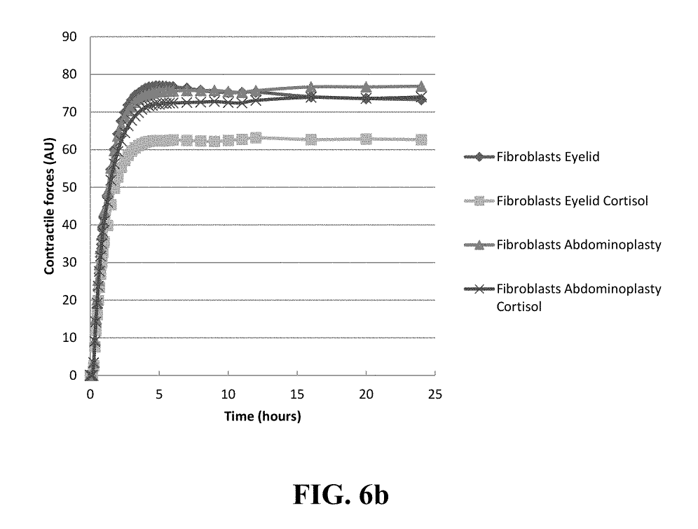

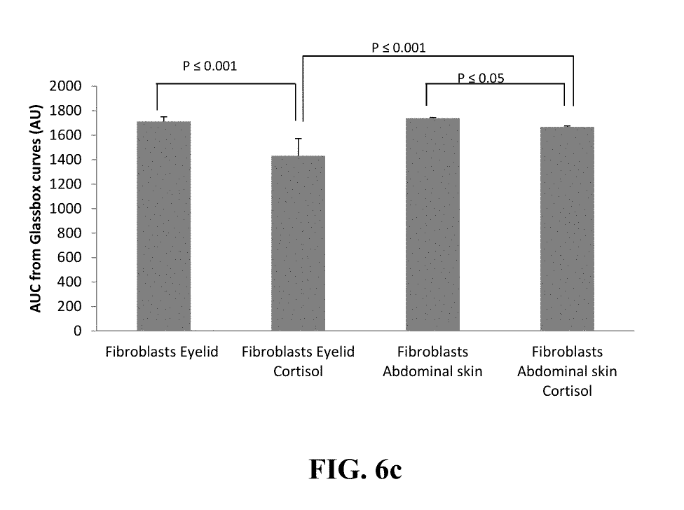

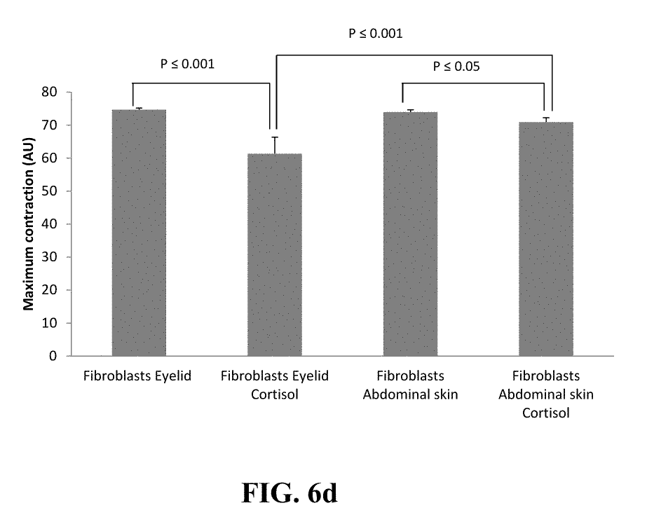

As shown in FIGS. 6a, 6b, 6c and 6d, exposure to cortisol induced a decrease in the contractile forces in both fibroblasts from eyelid and from abdominal tissue which was observed as a significant decrease of the area under the curve (AUC) and of maximum contractile force. The values of AUC and maximum of contraction were significantly lower in the presence of cortisol in fibroblasts from eyelid than in fibroblasts from abdominal skin. Differences may be due to, for example, within donor variation or may reflect body site differences.

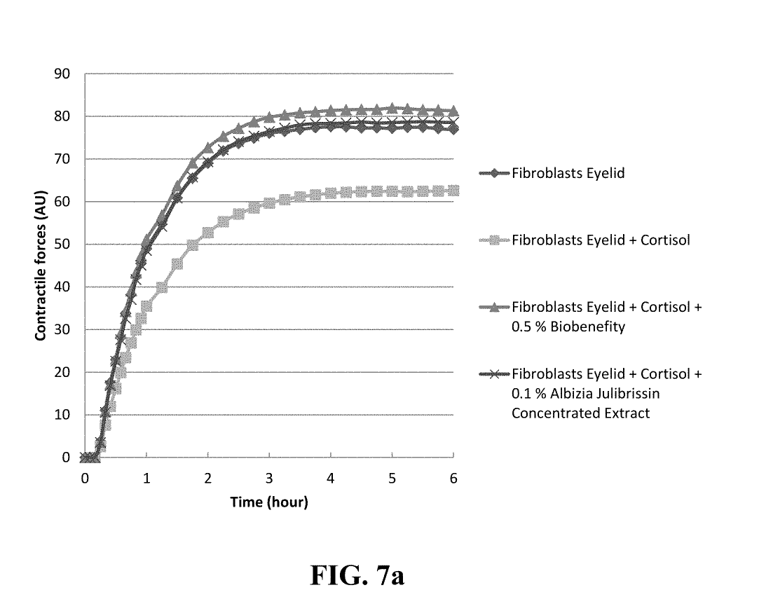

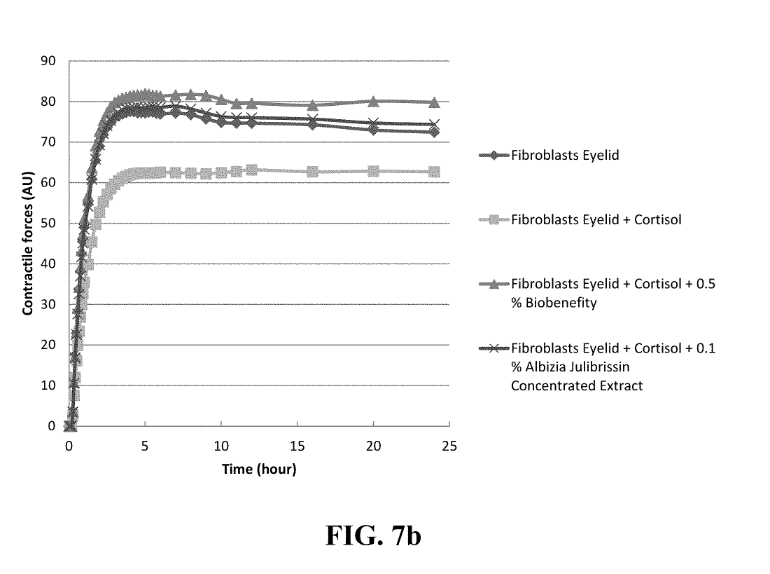

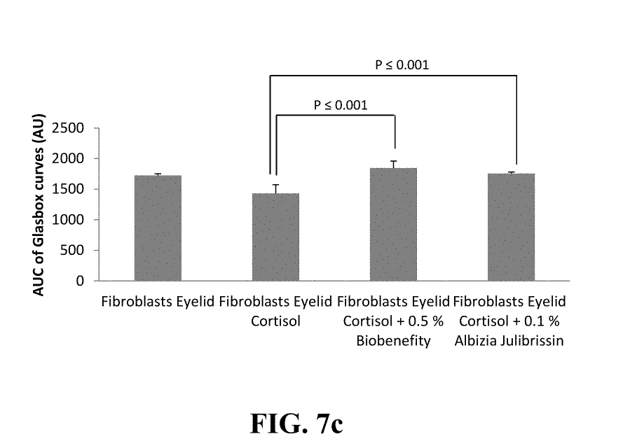

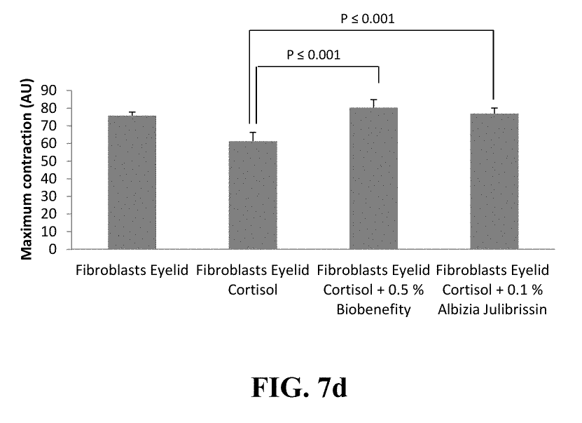

The effects of cosmetic raw materials, Biobenefity and Albizia julibrissin, on the contractile forces in fibroblasts from the eyelid, exerted on collagen lattices, were evaluated. Biobenefity was used at 0.5% w/v and Albizia julibrissin was used at 0.1% w/v, based on preliminary experiments performed to estimate a non-toxic concentration range for these materials (results not shown). As shown in FIGS. 7a, 7b, 7c and 7d, both Biobenefity and Albizia julibrissin protected against the cortisol-induced decrease of contractile forces. This effect was statistically significant. The Albizia julibrissin completely restored the contractile forces up to the level that was measured in the absence of cortisol. The activity of Biobenefity was observed to be stronger still, as the contractile forces observed were greater than the forces measured in the non-stressed fibroblasts. As it was also observed that there was no significant effect of these compounds on the contractile forces in the absence of cortisol (results not shown), it is theorized that, under these conditions, these compounds may not have a significant effect on the baseline contractile force values, but appear to offer significant protection in times of cellular stress.

### Example 3—Evaluation of Test Materials for Efficacy in Stimulating Contractile Forces of Fibroblasts Populated on Collagen Lattice

In this study, the GlaSbox® system was used to analyze the effect of cosmetic raw materials, TGFβ1, Taisoh Liquid B Jujube Extract (Ziziphus jujuba fruit), available from Ichimaru Pharcos, and Uplevity, available from Lipotec. TGFβ1, used in this study as a positive control, is known to play a role in cellular functions, including cell proliferation, differentiation, wound healing and matrix-related processes. Taisoh Liquid B Jujube Extract has been reported to stimulate wound healing. The inventors had previously observed that this compound stimulates collagen remodeling via phagocytosis, and was further shown to decrease the expression of the senescence marker p21 in H₂O₂-induced premature senescence in normal HDFs (data not shown) and in cortisol-induced premature senescence in ex vivo skin explants (see Example 1, hereinabove.) Uplevity is a tetrapeptide said to be designed to have an effect on the organization of elastic fibers so as to prevention of sagging or laxity of aging skin.

Fibroblasts were obtained from an abdominoplasty of a 51 year old woman. After thawing, cells were cultured in Dulbecco's modified Eagle's medium supplemented with 10% of fetal calf serum, 40 mg/l of gentamicin and 2 mg/l of fungizone (DMEMc), at 37° C., 5% CO2. Culture medium was changed twice a week.

### Preparation of Collagen Lattices Under Tension and Measurement of the Isometric Forces

Fibroblasts were embedded three-dimensionally in hydrated collagen gels composed of 6 volumes of 1.76× (DMEMc, NaHCO3, NaOH, antibiotics), 3 volumes rat tail type I collagen (2 mg/ml) and 1 volume of cellular suspension (8×10⁵ cells/ml) using a modified version of the technique developed by Bell et al. (Production of a tissue-like structure by contraction of collagen lattices by human fibroblasts of different proliferative potential in vitro. Proc. Natl. Acad. Sci. USA 76, 1274-1278 (1979). The lattice mixture was poured into the rectangular culture plate of the GlaSbox® and polymerized in less than 30 minutes at 37° C. Immediately after lattice formation TGFβ1, Taisoh liquid B Jujube extract or Uplevity was added in the cell culture medium. The GlaSbox® was then placed into a humidified incubator at 37° C., and force measurements were started after 30 minutes of stabilization, for 24 hours. The forces were expressed as arbitrary units (AU) after 24 hours of measurement.

### Calculations and Statistical Analysis

Each Glasbox® curve of contraction force versus time was fitted with GraphPad Prism® software to determine the area under the curve (AUC) and the maximal of contraction (Max). Area under the curve gives information on the global contraction force of fibroblasts during the experiment. Maximal contraction corresponds with the plateau of the fitted curve. Data were expressed as mean±standard deviation. The measurement of contractile forces was analyzed by means of a variance analysis with two factors (group versus control and time). This was followed by a Fisher post-hoc test. A p value less than 0.05 was considered significant.

### Results

### The Effect of TGF-β1 on the Contractile Forces Developed by Fibroblasts

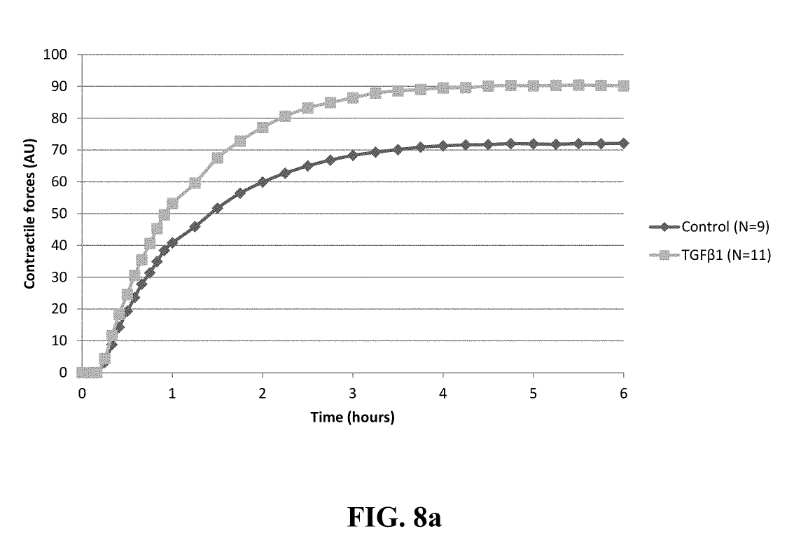

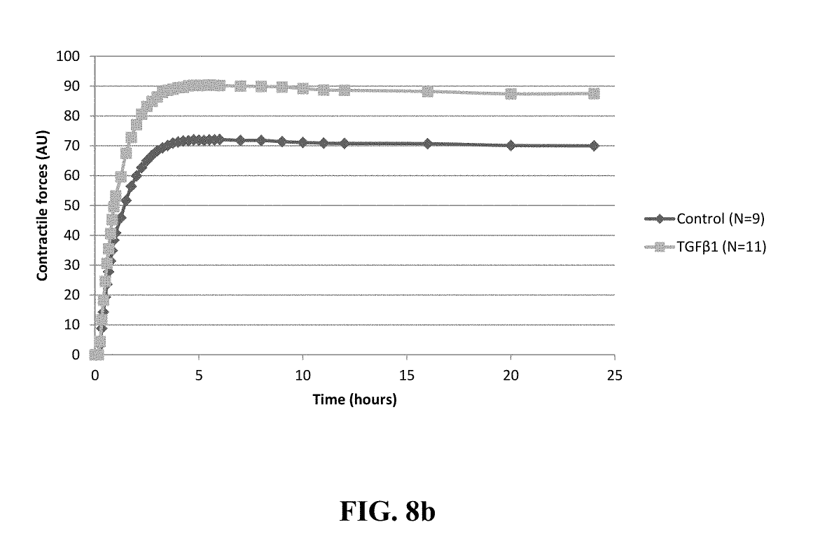

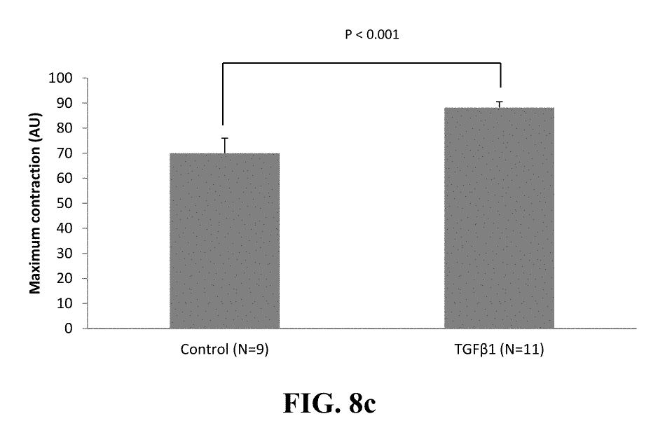

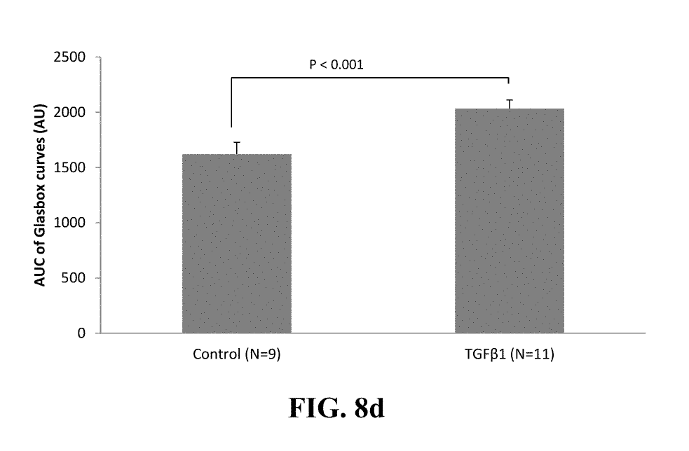

TGF-β1 is a secreted protein that is known to be involved in several cellular functions, including cell proliferation, differentiation and wound healing. FIG. 8a, 8b, 8c, 8d depicts the contractile forces of fibroblasts exerted on the collagen lattice as a function of time. Initially, there is observed an almost linear increase of the contractile forces. The maximum or plateau value is reached at about 3 hours. These results are similar to those described in Example 3, hereinabove. TGFβ1, used at 2.5 ng/ml, induced an increase of the contractile forces of fibroblasts. The overall effect of 2.5 ng/ml TGFβ on the contractile forces is represented in the AUC of the contraction force versus time curves. The contractile forces of fibroblasts treated with 2.5 ng/ml TGFβ increased by a statistically significant 25.5%. The maximum contraction of fibroblasts treated with 2.5 ng/ml TGFβ increased by a statistically significantly 26.0%.

### The Effect of Taisoh Liquid B Jujube Extract on the Contractile Forces Developed by Fibroblasts

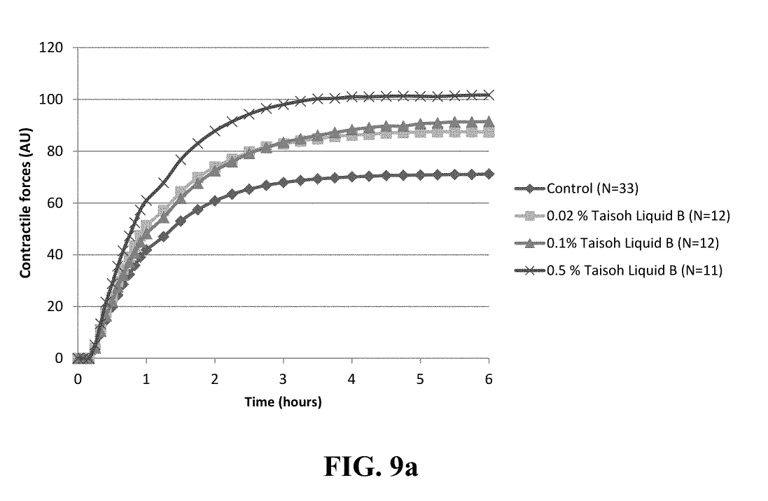

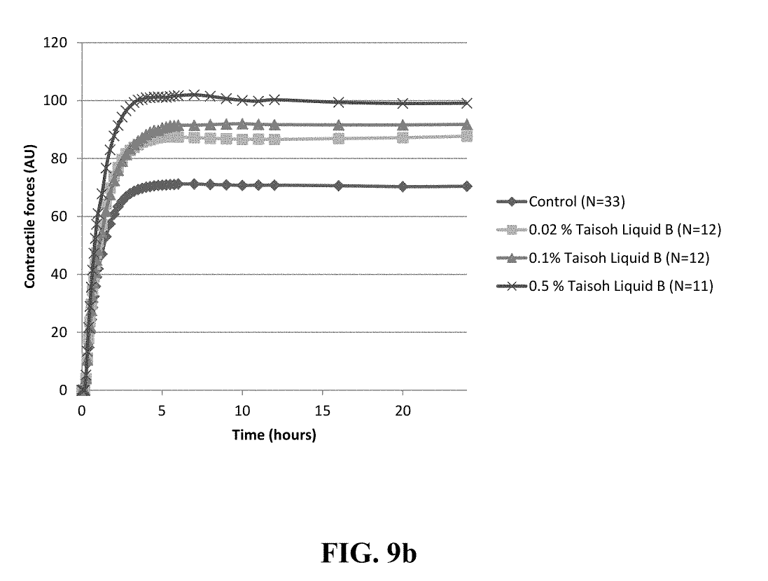

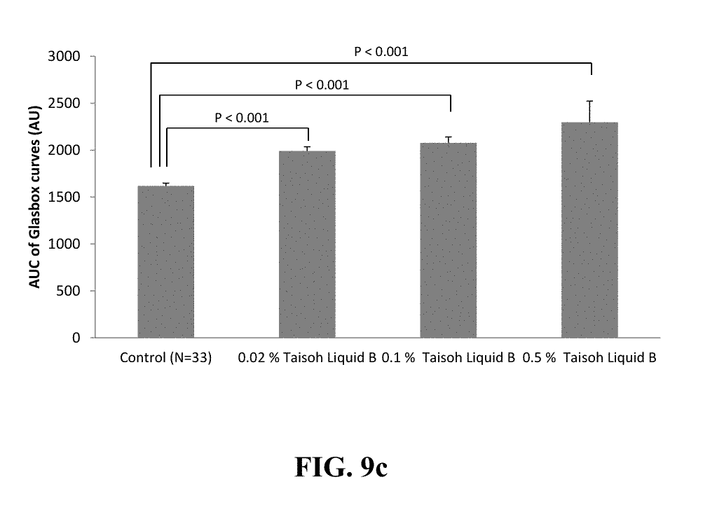

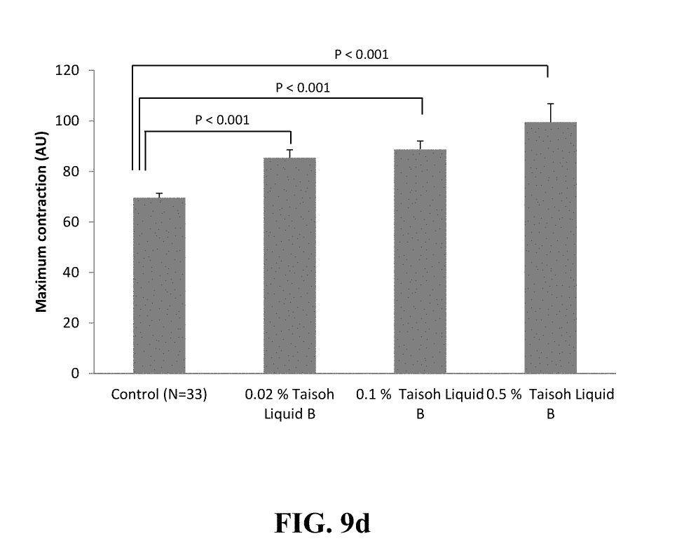

Primary experiments were performed to estimate a non-toxic concentration range of Taisoh Liquid B Jujube Extract (results not shown). Based on these data, the following concentrations of Taisoh Liquid B Jujube Extract were selected: 0.02%, 0.1% and 0.5% for testing. As shown in FIGS. 9a, 9b, 9c and 9d, Taisoh Liquid B Jujube Extract induced a dose dependent and statistically significant increase of the contractile forces of fibroblasts. The strongest effect was found at 0.5% Taisoh Liquid B Jujube Extract, which increased the AUC and the maximum contractile forces by 41.9% and 43.0% respectively.

### The Effect of Uplevity (Powder Version) on the Contractile Forces Developed by Fibroblasts

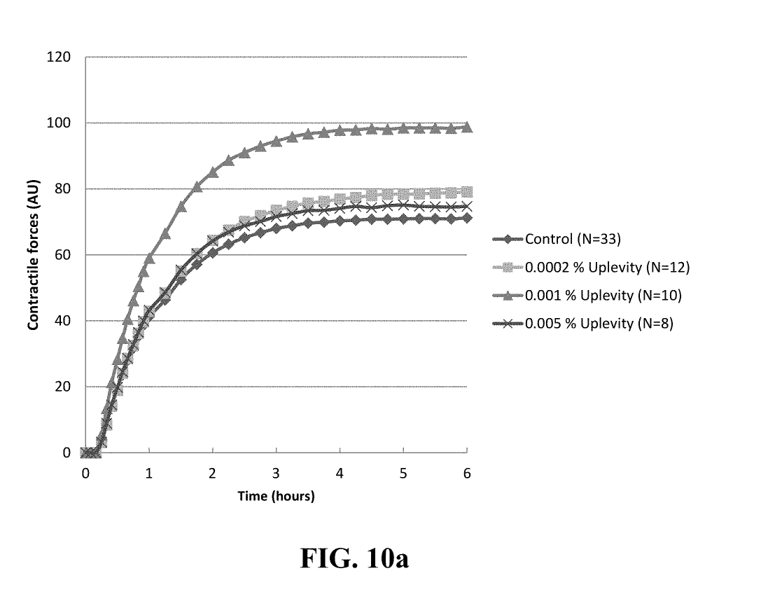

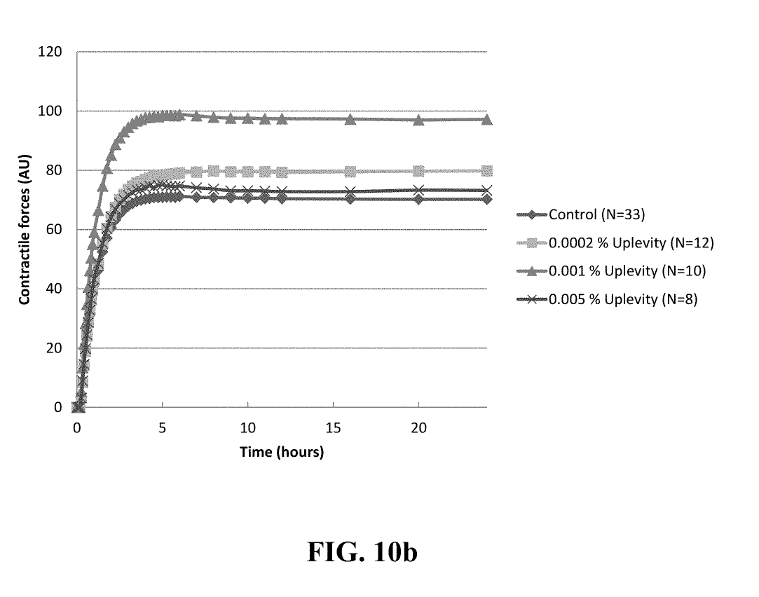

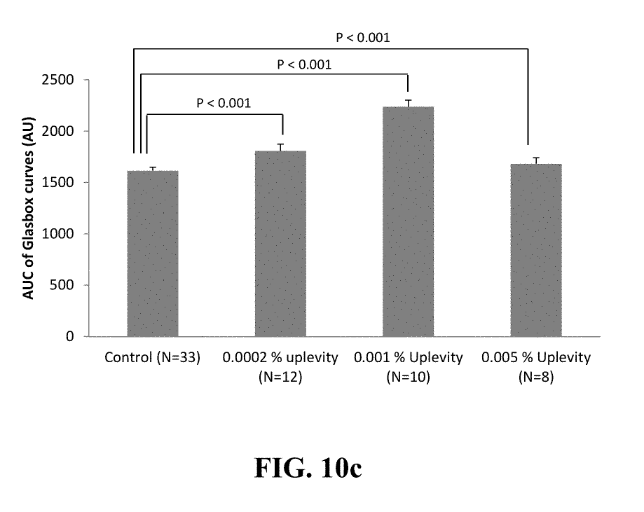

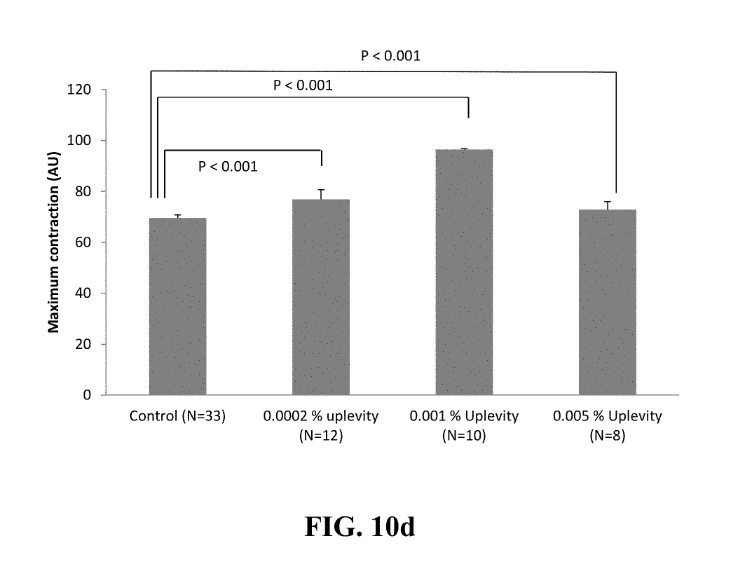

Primary experiments were performed to estimate a non-toxic concentration range of Uplevity (results not shown). Based on these data, following concentrations of Uplevity were selected: 0.0002% w/v, 0.001% w/v and 0.005% w/v for use. FIGS. 10a, 10b, 10c and 10d show the effect of Uplevity on the contractile forces of fibroblasts. Uplevity, used at 0.005-0.0002% w/v, increased the contractile forces at all concentrations. Under the current test conditions the strongest effect was found for the intermediate concentration of 0.001% w/v. The effect of Uplevity on the contractile forces is represented as the AUC of the contraction force versus time. A statistically significant increase of the AUC and maximum contractions were observed when the fibroblasts were treated with Uplevity. At 0.001% w/v, Uplevity induced the strongest effect with a 38.6% increase of the AUC and a 38.7% increase of the maximum contraction of the fibroblasts.

### Example 4—Evaluation of Test Materials for Efficacy in Reversing Cortisol-Induced Decrease in Contractile Forces of Fibroblasts Populated on Collagen Lattice

### Methods

### Cell Culture

Human dermal fibroblasts (HDFs, passages 6-7) were maintained in Falcon 75 cm² tissue culture flasks in DMEM supplemented with 10% FBS. Cells were harvested from monolayer culture, and placed in 6-well culture plates (1×10⁵ cells/well). Cells were pretreated with 25 μM hydrocortisone or with a combination of 25 μM hydrocortisone and different concentrations of test materials for 24 hours before being applied to free floating fibroblast populated collagen lattices.

### Preparation of Fibroblast-Populated Collagen Lattice (FPCL)

Gels containing collagen HDFs were prepared as described by Tomasek, J. J., et al. (Fibroblast contraction occurs on release of tension in attached collagen lattices: dependency on an organized actin cytoskeleton and serum. Anat. Rec., 1992, March; 232(3):359-68), incorporated herein by reference in its entirety, with modifications as follows. Briefly, collagen solution (Corning, Rat tail, 354236), concentrated DMEM, 0.1N NaOH and FBS, were gently mixed at 4° C., giving a suspension at a final density of 5×10⁵ cells and 3 mg/ml collagen. The collagen/cell suspension (2 ml total) was poured on 35 mm-uncoated dish and allowed to polymerize for 45 minutes at 37° C. Then lattices were released and gels were allowed to float in the medium. After 10 hours, the diameter of the collagen lattice of each dish was observed.

### Measurement of Gel Contraction

Fibroblast contractility was assessed by measuring changes in the surface area of collagen I gels mediated by fibroblasts. After polymerization, lattices were released with a pipette tip and gels were incubated for 10 hours. Thereafter, lattice diameter was measured. The effect of the fibroblasts on contraction of the gels (i.e., promotion or inhibition) is represented as the area of the contracted matrix as a percentage of the initial gel.

Promotion rate (%)=(πa²−πc²)−(πa²−πb²)/(πa²−πb²)*100%

Inhibition rate (%)=[(πa2−πe2)−(πa2−πb2)−(πa2−πb2))]/((πa2−πd2)−(πa2−πb2))*100%

Triplicate FPCL were cast for each test and control group and all experiments were repeated three times.

### Statistical Analysis

An analysis of variance (ANOVA) and Student's t test were used for comparison among groups. P-values of less than 0.05 was considered to be significant.

### Results

To determine the effect of various test materials on the contraction of floating collagen gels populated with fibroblasts, cells were pretreated with various concentrations of test materials, cast into the floating collagen gels (collagen lattices), and then left undisturbed for 10 hours.

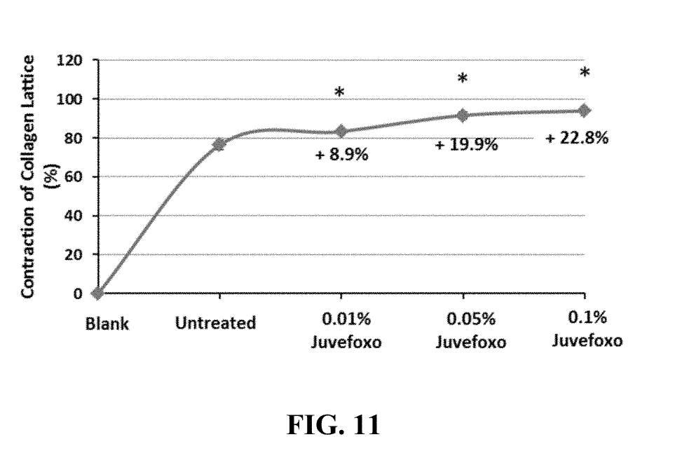

FIG. 11 depicts the area changes of the lattice upon treatment with or without 0.01%, 0.05% or 0.1% Juvefoxo, which contains acetyl hexapeptide-50. The contractions of the lattices were significantly increased by 8%, 19% and 22%, respectively, as compared with the untreated lattices. The values represent percent contraction of the gel in comparison with the initial non-contracted ones, are the mean of three independent experiments performed in triplicate. Error bars correspond to standard deviations (*p<0.05).

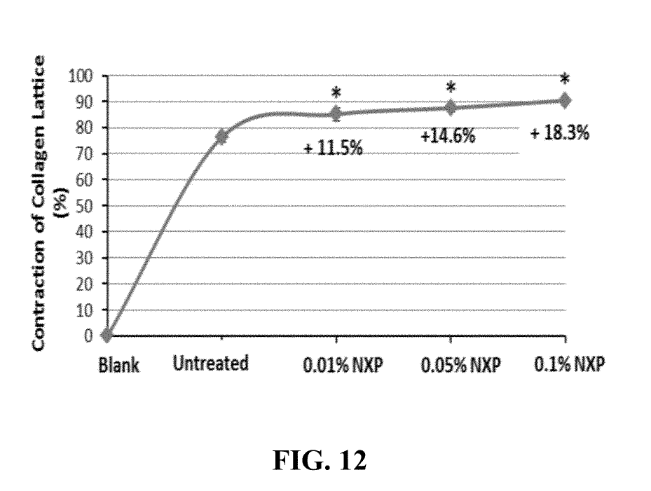

FIG. 12 depicts the area changes of the lattices populated with control fibroblasts or with fibroblasts pretreated with 0.01%, 0.05% or 0.1% NXP, containing whey protein. Contractions of the lattices were increased by 11.4%, 14.6% and 18.3%, respectively, compared with that of the untreated lattices. Values representing percent contraction of the gel lattices in comparison with the untreated (non-contracted) gel lattices are the mean of three independent experiments performed in triplicate. Error bars correspond to standard deviations (*p<0.05).

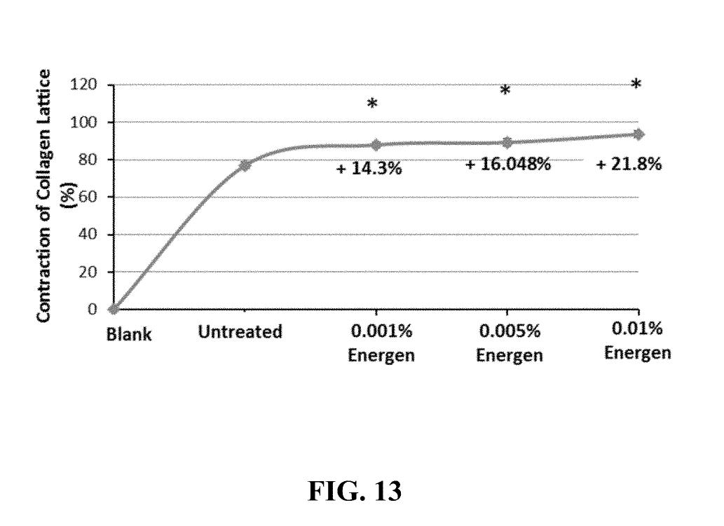

As indicated in FIG. 13, fibroblasts pretreated with Energen, containing Sapindus mukurossi fruit extract and Caesalpinia spinosa gum, showed powerful promoting effects by fibroblasts on collagen gel contraction in a dose-dependent manner; 0.001%, 0.005%, and 0.01% Energen increasing the effect of contraction by 14%, 16% and 21.8%, respectively. Values representing percent contraction of the gel lattices in comparison with the untreated, non-contracted lattices are the mean of three independent experiments performed in triplicate. Error bars correspond to standard deviations (*p<0.05).

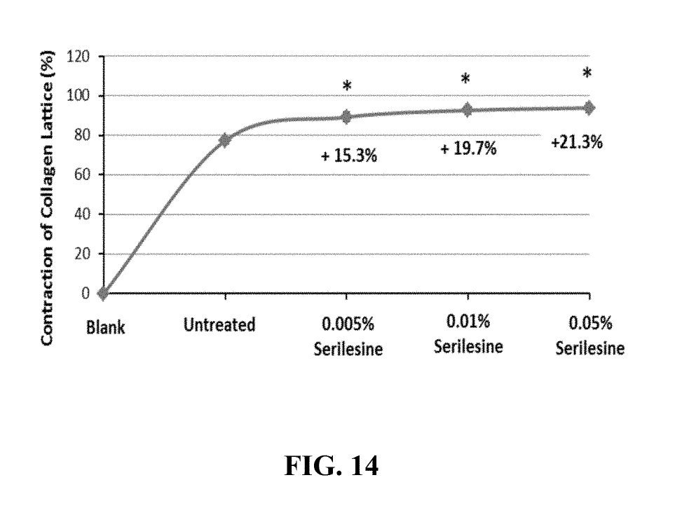

As indicated in FIG. 14, fibroblasts pretreated with Serilesine, containing hexapeptide-10, also effected a significant increase in contraction of the collagen lattices. Serilesine, at 0.005% promoted a 15.3% increase in contraction by fibroblasts, while the promoting effect increased to 21.3% when fibroblasts were was pretreated with 0.05% Serilesine, compared with that of untreated-lattice. Values representing percent contraction of the gel lattices in comparison with the untreated, non-contracted lattices are the mean of three independent experiments performed in triplicate. Error bars correspond to standard deviations (*p<0.05).

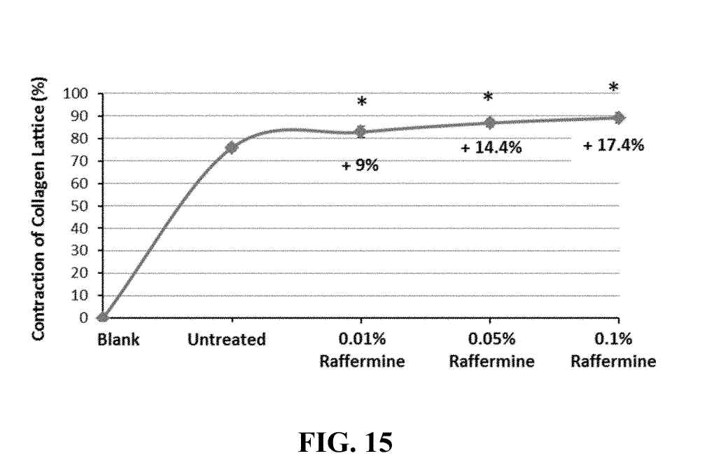

Fibroblasts treated with Raffermine, containing hydrolyzed soy flour, also effected an increase in contraction of collagen lattices; fibroblasts treated with 0.05% and 0.1% Raffermine-boosting lattice contraction by 14% and 17.4%, respectively, compared with untreated lattices FIG. 15 values, representing percent contraction of the gel in comparison with the initial non-contracted one, are the mean of three independent experiments performed in triplicate. Error bars correspond to standard deviations. (*p<0.05).

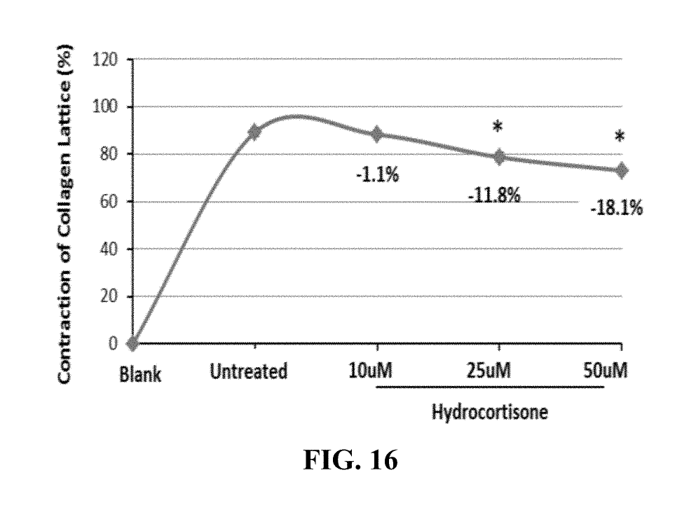

As indicated in FIG. 16, when exposed to fibroblasts treated with different concentrations of hydrocortisone, contraction of collagen lattices was inhibited. Compared with untreated lattices, fibroblasts treated with 25 μM and 50 μM hydrocortisone caused significant decrease of contraction by 11.8% and 18%, respectively. Values representing percent contraction of the gel in comparison with the initial non-contracted one are the mean of three independent experiments performed in triplicate. Error bars correspond to standard deviations. (*p<0.05).

Test materials which had been screened for efficacy in promoting fibroblast contractility were further evaluated for their ability to protect against the inhibitory effect of hydrocortisone on lattice contractility. As indicated below, Juvefoxo, NXP and Energen were shown to reverse the inhibitory effect of 25 μM hydrocortisone on collagen lattices when fibroblasts were pretreated with the combination of hydrocortisone and the test material prior to the fibroblasts being cast onto floating collagen lattices.

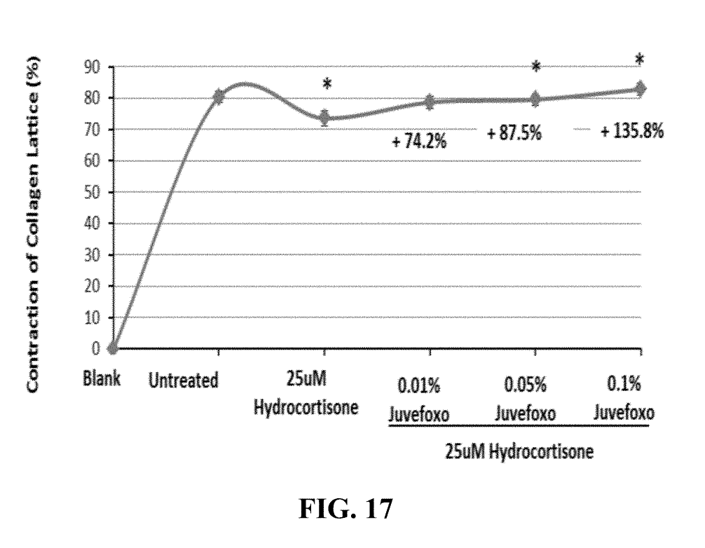

Juvefoxo, used at 0.01%, 0.05% and 0.1% was demonstrated to counteract the contraction inhibited by hydrocortisone from 74% to 135.8%, compared with a 9% decrease in contraction induced by hydrocortisone, as shown in FIG. 17. Values representing percent contraction of the gel in comparison with the initial non-contracted one are the mean of three independent experiments performed in triplicate. Error bars correspond to standard deviations. (*p<0.05)

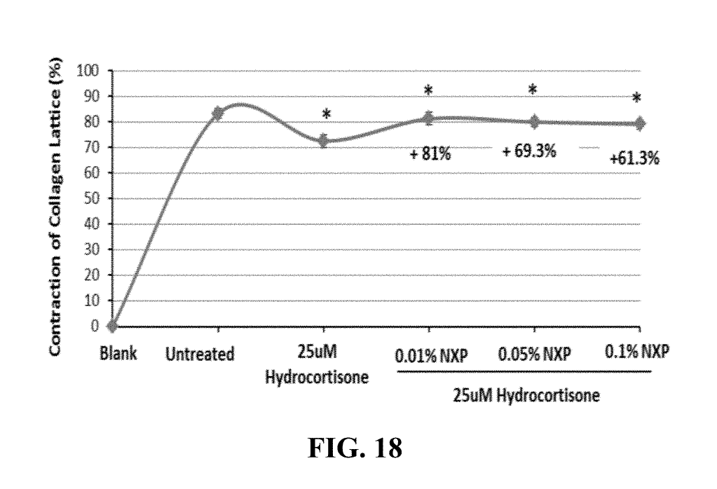

The use of NXP at 0.01% resulted in an 81% reverse of the effects of hydrocortisone, and at 0.1%, NXP not only reversed the effects of the hydrocortisone but promoted an increase in contractility of 61.3%, over the level of contractility effected by hydrocortisone FIG. 18. Values representing percent contraction of the gel in comparison with the initial non-contracted one are the mean of three independent experiments performed in triplicate. Error bars correspond to standard deviations (* p<0.05).

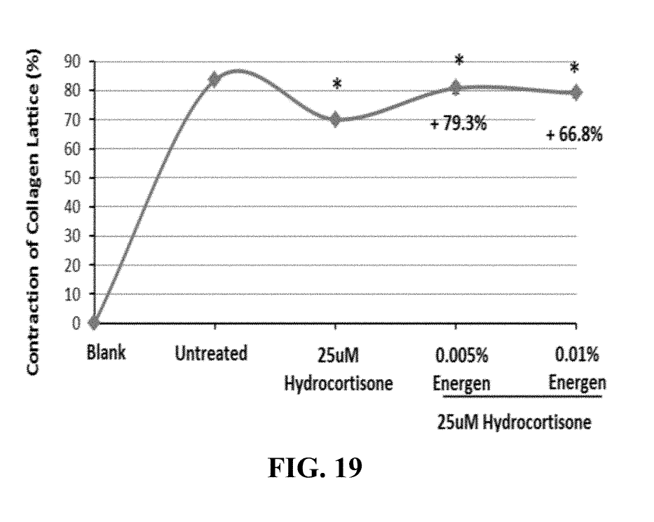

Energen, used at 0.005% and 0.01%, also resulted in a reversal of the effects of hydrocortisone, stimulating an increase of contraction of gel lattices by 79.3% and 66.8%, respectively, compared with the inhibitory effect of hydrocortisone FIG. 19. Values, representing percent contraction of the gel in comparison with the initial non-contracted one, are the mean of three independent experiments performed in triplicate. Error bars correspond to standard deviations (*p<0.05).

### Example 5—Effect of Actives on Elastin Release by HDFs in an In Vitro Model

### Methods

HDFs, at passage 4, were plated in 96 well plates. After the cells reached confluency they were placed under starvation conditions for 48 hours. Cells then were treated with test materials in cell medium for 72 hours after which the medium was collected for analysis of elastin (Elastin Elisa assay, SOP D.33). Cell viability also was measured using the MTT assay (SOP D.29). Statistical analysis was performed with an ANOVA+Fisher LSD post hoc test. A p value of less than 0.05 was considered significant.

### Results

The results are presented as pg/ml elastin corrected for viability. The percent increase in elastin release is calculated as:

% increase=[Amount Elastin_{active}/Amount Elastin_{control}]×100−100

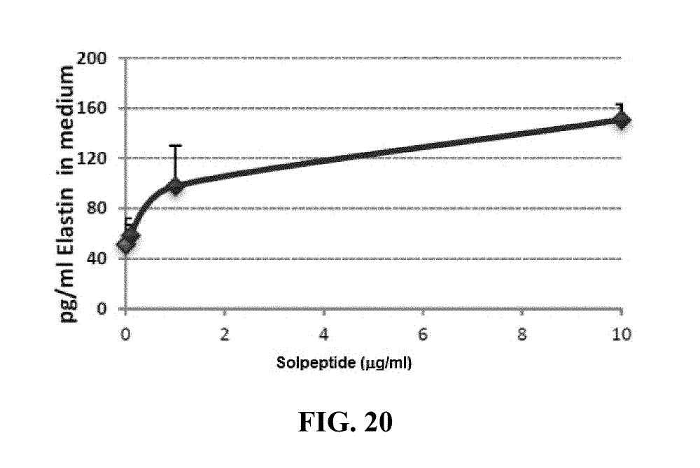

FIG. 20 shows that Solpeptide (Solanum tuberosum) increased the elastin release in a dose dependent manner. At the highest concentration of 10 μg/ml, a significant increase of 193% was detected (p<0.01) compared with untreated cells.

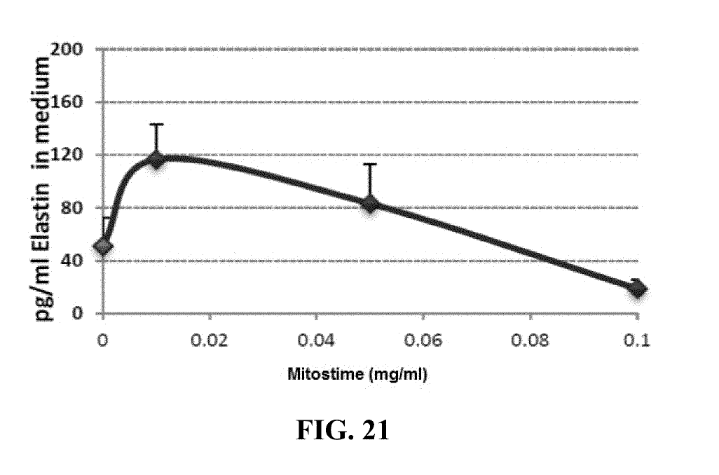

FIG. 21 shows that Mitostime increased the elastin synthesis. At a concentration of 0.01 mg/ml, a significant increase of 127% was measured (p<0.01) compared with untreated cells. However, at higher concentrations the elastin levels were reduced.

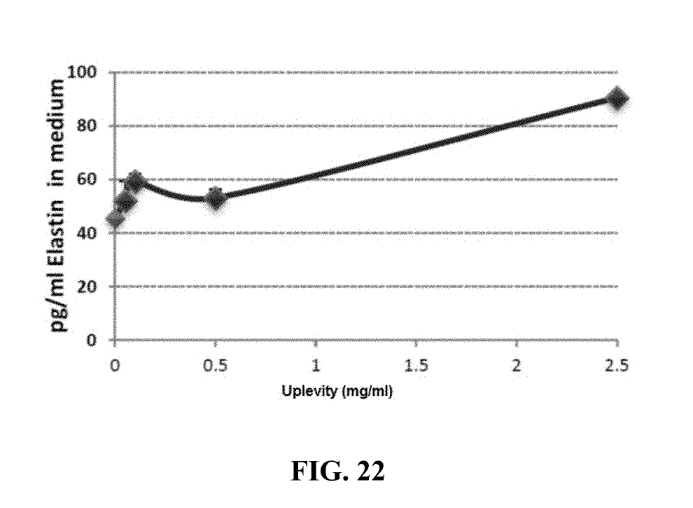

FIG. 22 shows that Uplevity increased the elastin synthesis in a dose dependent manner. At the highest concentration of 2.5 mg/ml, a significant increase of 99% was measured (p<0.01) compared with untreated cells.

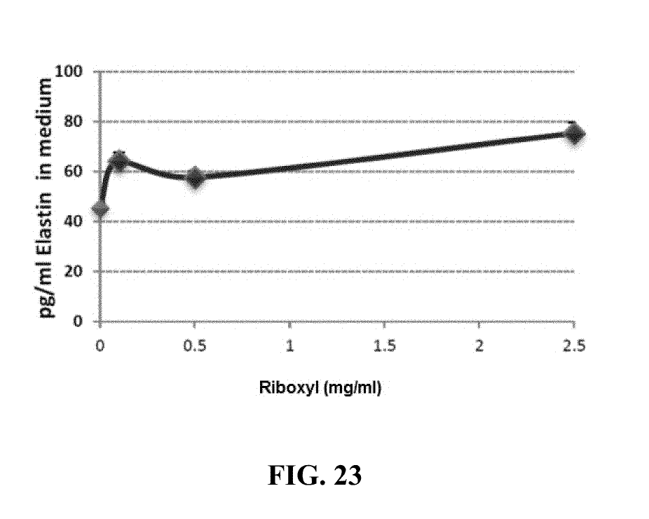

FIG. 23 shows that Riboxyl (D-ribose), said to enhance the elasticity of skin and preventing wrinkles by stimulating synthesis of structuring macromolecules of the dermis, including collagen, fibronectin, elastin, hyaluronic acid, increased the elastin synthesis in a dose dependent manner. At the highest concentration of 2.5 mg/ml, a significant increase of 66% (p<0.01) was measured compared with untreated cells.

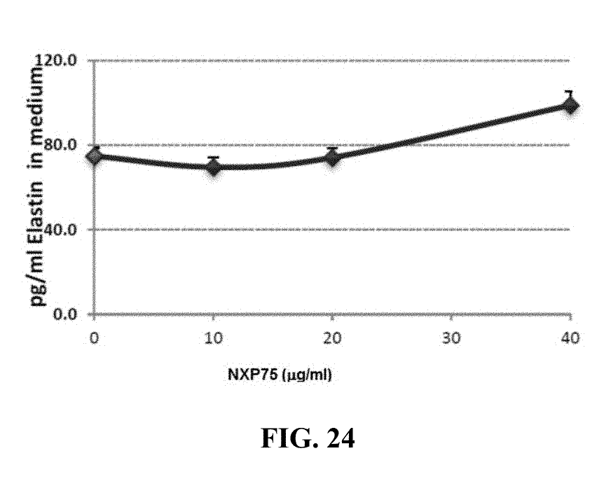

FIG. 24 shows that 40 μg/ml whey protein NXP75 significantly increased elastin synthesis by 32% (p<0.01) compared with untreated cells.

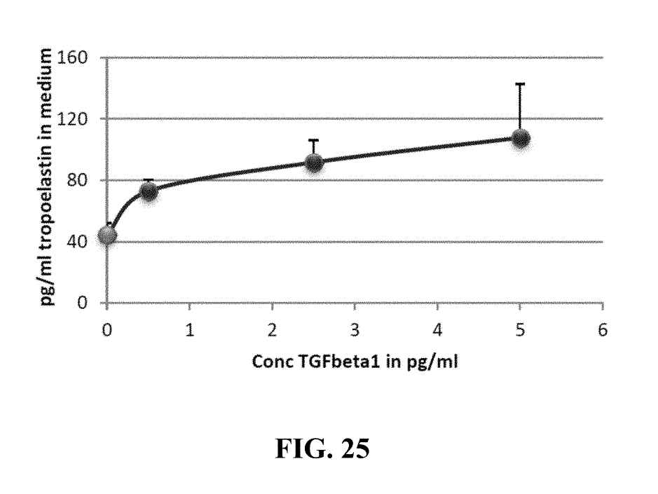

FIG. 25 demonstrates that TGFβ1 increased tropoelastin synthesis (correlated with elastin release) in a dose response manner. At the highest concentration of 5 pg/ml TGFβ1, a significant increase of 175% in tropoelastin synthesis was observed (p<0.01) compared with untreated cells.

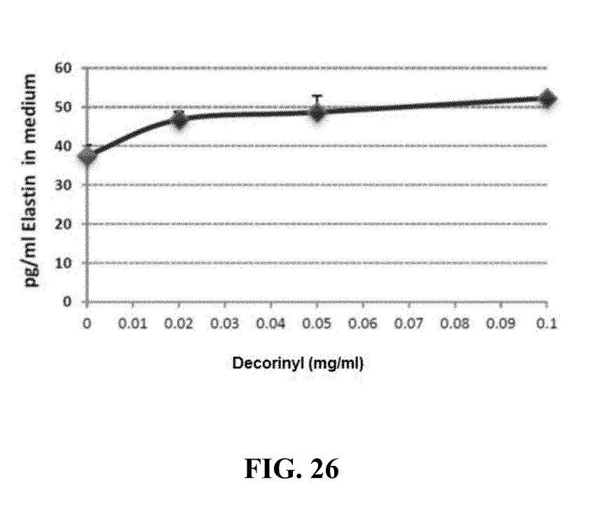

FIG. 26 shows that Decorinyl (a tetrapeptide said to mimic the activity of Decorin, a proteoglycan that binds to collagen fibers and controls their diameter resulting in more toned skin) increased the elastin synthesis in a dose dependent manner. At the highest concentration of 0.1 mg/ml, a significant increase of 39% (p<0.01) was detected.

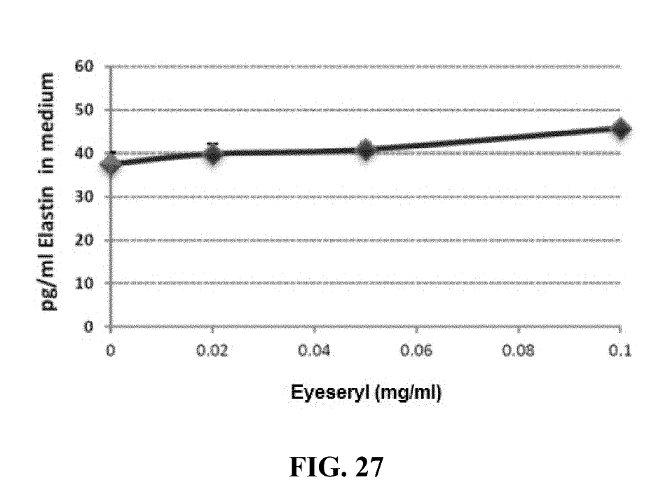

FIG. 27 shows that Eyeseryl (a tetrapeptide said to to prevent loss of elasticity) increased the elastin synthesis. At the highest concentration of 0.1 mg/ml, a significant increase of 22% was detected (p<0.01).

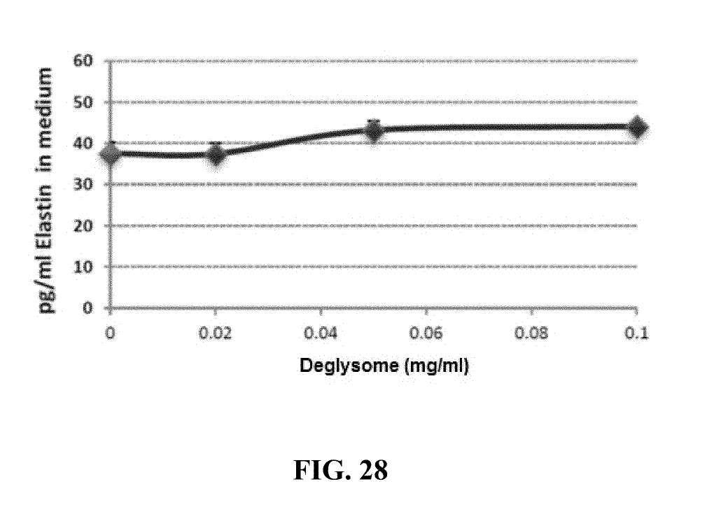

FIG. 28 shows that Deglysome LYO (containing algae galactan, and said to limit cellular and tissue damage caused by glycation which is recognized to impair functioning of biomolecules) increased the elastin synthesis. At the highest concentration of 0.1 mg/ml, a significant increase of 17% was detected (p<0.01).

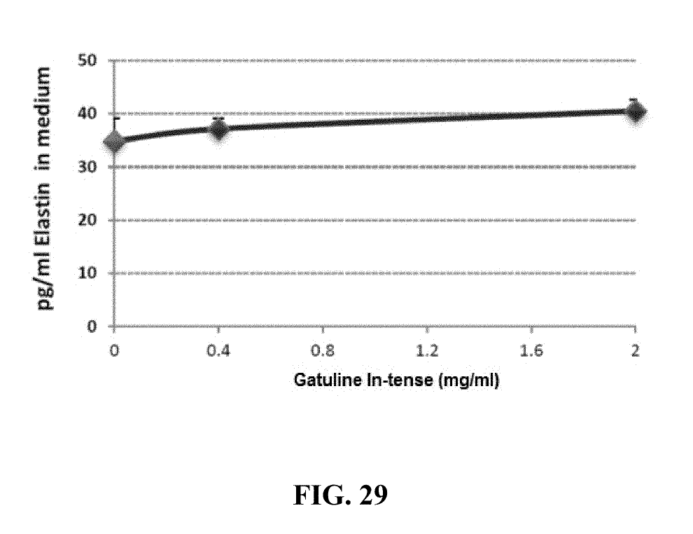

FIG. 29 shows that Gatuline In-tense (caprylic/capric triglyceride (and) Spilanthes acmella flower extract, said to target loss of skin firmness and appearance of deep wrinkles by stimulating fibroblast biomechanical function, boosting interaction between collagen fibers and fibroblasts to reorganize dermis structure an tighten skin from within) increased the elastin synthesis. At the highest concentration of 2 mg/ml, a significant increase of 16% was detected (p<0.01).

### Example 6—Evaluation of Test Materials for Efficacy in Stimulating Fibrillin Synthesis in In Vitro Human Dermal Fibroblasts (HDFs) Cell Culture Model

Fibrillins, glycoproteins secreted by fibroblasts, are essential for the formation of elastic fibers found in connective tissue. Test compounds Mitostime (extract of Laminaria digitata) and Milk Peptide Complex (MPC or whey protein, available from CLR, Germany) were evaluated for their capacity to stimulate the synthesis of fibrillin-1 in HDFs.

### Method

An aliquot of a selected fibroblast cell line (HDFs) was thawed, placed into culture, and allowed to establish good growth before passaging into a 24-well plate (5×10⁴ cells/1 ml well). After overnight adhesion to the well, test compounds were added to the medium at three different concentrations and the cells were incubated for 24 hours. A positive control of 100 ng/ml TGFβ1, was included. The negative controls used were 0.1% BSA and 0.1% EtOH. After 24 hours, medium was harvested, centrifuged, and transferred to fibrillin-1 sandwich ELISA plates to determine the amount of fibrillin released.

### Results

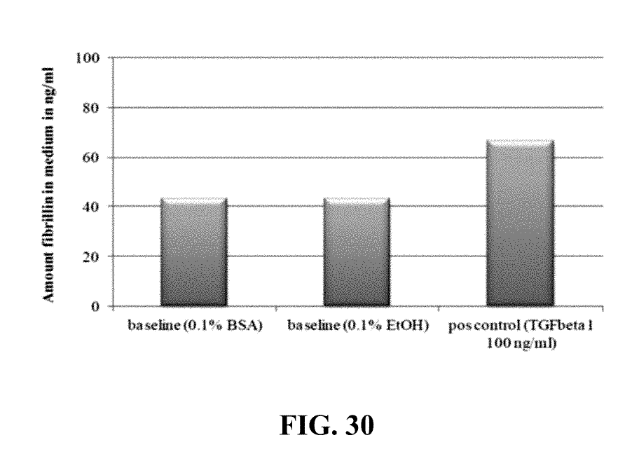

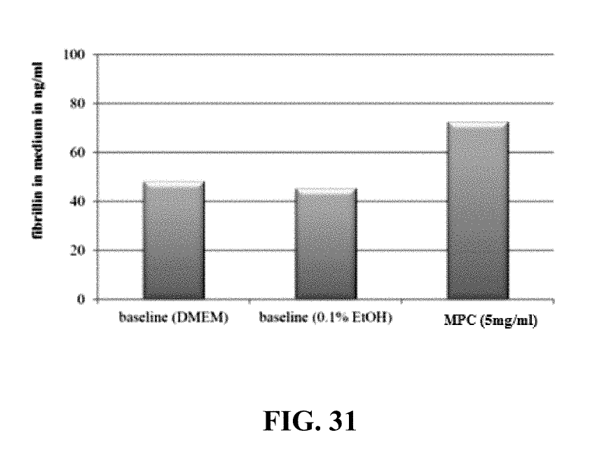

A baseline level (no added stress) of about 45 ng/ml fibrillin release was detected from the cells (DMEM sample). The presence of ethanol (0.1%) was not found to affect the baseline release. Treatment of the cells with MPC for 24 hours was found to stimulate the fibrillin release by 60%, as indicated in FIG. 30. Mitostime, tested at 5 mg/ml, was observed to stimulate fibrillin release with an increase of about 38%, as shown in FIG. 31.

### CONCLUSION

TGFβ1, and actives MPC and Mitostime, were found to stimulate the fibrillin release at baseline level.

Although the invention has been variously disclosed herein with reference to illustrative embodiments and features, it will be appreciated that the embodiments and features described hereinabove are not intended to limit the scope of the invention, and that other variations, modifications and other embodiments will suggest themselves to those of ordinary skill in the art. The invention therefore is to be broadly construed, consistent with the claims hereafter set forth.

What is claimed is:

1. A method for identifying a material having an efficacy for reversing a stress-induced premature senescence phenotype associated with the appearance of fatigued skin, the method comprising
(a) providing a dermal equivalent skin model;
(b) incubating the dermal equivalent skin model of (a) with a stress-inducing ingredient in an amount and for a time sufficient to induce a premature senescence phenotype in the dermal equivalent skin model;
(c) incubating the dermal equivalent skin model of (b) with a test material; and
(d) ascertaining whether the test material has an efficacy for reversing the premature senescence phenotype in the dermal equivalent skin model.

2. A method for identifying a material having an efficacy for preventing or minimizing development of a premature senescence phenotype associated with the appearance of fatigued skin, the method comprising:
(a) providing a dermal equivalent skin model;
(b) treating the dermal equivalent skin model of (a) with a test material;
(c) treating the dermal equivalent skin model of (b) with a stress-inducing ingredient in an amount and for a time sufficient to have induced a premature senescence phenotype in a dermal equivalent skin model in the absence of the test material; and
(d) ascertaining whether the test material has an efficacy for preventing or minimizing development of the premature senescence phenotype in the dermal equivalent skin model.

3. The method of claim 1, wherein the dermal equivalent skin model is an in vitro model comprising human dermal fibroblasts (HDFs), an ex vivo model comprising HDFs, or a fibroblast populated collagen lattice.

4. The method of claim 2, wherein the dermal equivalent skin model is an in vitro model comprising human dermal fibroblasts (HDFs), an ex vivo model comprising HDFs, or a fibroblast populated collagen lattice.

5. The method of claim 1, wherein the premature senescence phenotype is characterized by presence of a biomarker selected from an increase in expression of p21 in fibroblasts, an increase in expression of progerin in fibroblasts, a decrease in elastin production in fibroblasts, a decrease in fibrillin production in fibroblasts, a decrease in fibroblast contractility, a decrease in number of skin layers, or a combination of any two or more thereof.

6. The method of claim 2, wherein the premature senescence phenotype is characterized by presence of a biomarker selected from an increase in expression of p21 in fibroblasts, an increase in expression of progerin in fibroblasts, a decrease in elastin production in fibroblasts, a decrease in fibrillin production in fibroblasts, a decrease in fibroblast contractility, a decrease in number of skin layers, or a combination of any two or more thereof.

7. The method of claim 1, wherein fatigued skin is characterized by one or more of wrinkles on the skin, hyperpigmented skin, loss of subcutaneous fat, skin laxity, and reduced skin radiance.

8. The method of claim 2, wherein fatigued skin is characterized by one or more of wrinkles on the skin, hyperpigmented skin, loss of subcutaneous fat, skin laxity, and reduced skin radiance.

9. The method of claim 1, wherein the stress-inducing ingredient is cortisol.

10. The method of claim 2, wherein the stress-inducing ingredient is cortisol.

11. The method of claim 1, wherein the dermal equivalent skin model of step (b) is incubated with the stress-inducing ingredient in an amount in the range of from about 0.000001 to about 5 weight % and for a time in the range of from about 1 hour to about 24 hours.

12. The method of claim 1, wherein the dermal equivalent skin model of step (c) is incubated with the test material in an amount in the range of from about 0.0001% to about 5% weight %, and for a time in the range of from about 1 hour to about 7 days.

13. The method of claim 2, wherein the dermal equivalent skin model of step (b) is incubated with the test material in an amount in the range of from about 0.0001% to about 0.5 weight %, and for a time in the range of from about 1 hour to about 7 days.

14. The method of claim 2, wherein the dermal equivalent skin model of step (c) is incubated with the stress-inducing ingredient in an amount in the range of from about 0.000001% to about 5 weight %, and for a time in the range of from about 1 hour to about 24 hours.

15. A composition for preventing, minimizing or reversing a biological impact of stress on skin, the composition comprising a combination of:
(a) at least one cosmetic material demonstrating an efficacy for protecting against or reversing development of a stress-induced premature senescent phenotype associated with fatigued skin; and
(b) at least one cosmetic material demonstrating an efficacy for rebuilding epidermis; wherein the combination of (a) and (b) results in restored elasticity in the skin.

16. The composition of claim 15, wherein the biological impact of stress on skin is characterized by one or more of wrinkles in skin, hyperpigmented skin, skin laxity, reduced presence of subcutaneous fat and reduced skin radiance.

17. The composition of claim 15, wherein the premature senescent phenotype is characterized by one or more of enhanced expression of p21 or progerin in fibroblasts, decreased fibroblast contractility, decreased elastin production in fibroblasts, decreased fibrillin production in fibroblasts, and a reduced number of skin layers.

18. The composition of claim 15, wherein the at least one cosmetic material (a) demonstrates an efficacy for one or more of:
(1) preventing or reversing increased expression of p21 or progerin in fibroblasts,
(2) preventing or reversing decreased fibroblast contractility,
(3) preventing decreased elastin production in fibroblasts,
(4) preventing decreased fibrillin production in fibroblasts, and
(5) preventing or reversing a decreased number of skin layers;
wherein the at least one cosmetic material (b) demonstrates an efficacy for one or both of increasing synthesis of elastin and increasing synthesis of fibillin.

19. A method for improving the appearance of fatigued skin, the method comprising
(a) applying to skin in need of such improvement at least one cosmetic material demonstrating an efficacy for protecting against or reversing development of a stress-induced premature senescent phenotype associated with appearance of fatigued skin; and
(b) applying to skin in need of such improvement at least one cosmetic material demonstrating an efficacy for rebuilding epidermis; wherein (a) and (b) may be applied to skin simultaneously or sequentially in any order to restore elasticity to the skin.

20. The method of claim 19, wherein step (a) comprises applying to the skin a cosmetic material demonstrating an efficacy for one or more of:
(1) preventing or reversing increased expression of p21 or progerin in fibroblasts,
(2) preventing or reversing decreased fibroblast contractility,
(3) preventing decreased elastin production in fibroblasts,
(4) preventing decreased fibrillin production in fibroblasts, and
(5) preventing or reversing a decreased number of skin layers;
wherein step (b) comprises applying to the skin a cosmetic material demonstrating an efficacy for one or both of increasing synthesis of elastin and increasing synthesis of fibillin.
